(19) United States
(12) Patent Application Publication
(10) Pub. No.: US 2026/0071213 A1
(43) Pub. Date: Mar. 12, 2026

(54) COMPOSITIONS TARGETING HBG1 AND HBG2 AND METHODS OF USE THEREOF

(71) Applicant: Poseida Therapeutics, Inc., San Diego, CA (US)

(72) Inventors: Haibin XI, San Diego, CA (US); Patrick Won-Pyo HONG, San Diego, CA (US); Blair B. Madison, San Diego, CA (US)

(21) Appl. No.: 19/389,774

(22) Filed: Nov. 14, 2025

Related U.S. Application Data
(63) Continuation of application No. PCT/US2024/029750, filed on May 16, 2024.
(60) Provisional application No. 63/502,903, filed on May 17, 2023.

(51) Int. Cl. C12N 15/11; A61K 31/7088; A61K 38/46; C12N 9/22; C12N 15/88; C12N 15/90

(52) U.S. Cl. CPC C12N 15/11; A61K 31/7088; A61K 38/465; C12N 9/226; C12N 15/88; C12N 15/907; C07K 2319/00; C12N 2310/20

(57) ABSTRACT

Disclosed are methods and compositions for functional genetic modifications at selected genomic sites such as BCL11A gene, HMG1 promoter and/or HMG2 promoter. Also provided are cell populations, which comprise the functional genetic modification at one or more selected gene loci.

### CROSS-REFERENCE TO RELATED APPLICATIONS

This application is a continuation of International Patent Application No. PCT/US2024/029750, filed May 16, 2024, which claims benefit of U.S. Provisional Application No. 63/502,903, filed May 17, 2023. Each of the foregoing applications is incorporated herein by reference in its entirety.

### FIELD OF THE DISCLOSURE

The present disclosure is directed to the field of genetic editing and genomic engineering. More particularly, the present disclosure is directed to compositions and methods for targeted genetic modification and modulating expression of a target nucleic acid sequence and applications thereof.

### INCORPORATION-BY-REFERENCE OF SEQUENCE LISTING

The instant application contains a Sequence Listing which has been submitted in XML format via Patent Center and is herein incorporated by reference in its entirety. Said XML copy, created on Nov. 14, 2025, is named “000218-0145-101-SL.xml” and is 61,670 bytes in size.

### BACKGROUND OF THE DISCLOSURE

Genome editing refers to strategies and techniques for the targeted, specific modification of the genetic information (genome) of living organisms. Genome engineering is an active field of research because of the wide range of possible applications, particularly in the area of human health, e.g., to correct a gene carrying a harmful mutation or to explore the function of a gene. Early technologies developed to insert a transgene into a living cell were often limited by the random nature of the insertion location of the new sequence into the genome. Common genome editing strategies allow a specific area of the DNA to be modified, thereby increasing precision of the correction or insertion compared to earlier technologies. While these platforms offer a greater degree of reproducibility and decreased level of unintended effects from random insertions and deletions in the genome, limitations remain.

Hemoglobin (Hb) carries oxygen in erythrocytes or red blood cells (RBCs) from the lungs to tissues. During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. The average adult makes less than 1% HbF out of total hemoglobin. The α-hemoglobin gene is located on chromosome 16, while the β-hemoglobin gene (HBB), A gamma (γA)-globin chain (HBG1, also known as gamma globin A), and G gamma (γG)-globin chain (HBG2, also known as gamma globin G) are located on chromosome 11 within the globin gene cluster (also referred to as the globin locus).

Mutations in HBB can cause hemoglobin disorders (i.e., hemoglobinopathies) including sickle cell disease (SCD) and beta-thalassemia (β-Thal). Approximately 93,000 people in the United States are diagnosed with a hemoglobinopathy. Worldwide, 300,000 children are born with hemoglobinopathies every year. Because these conditions are associated with HBB mutations, their symptoms typically do not manifest until after globin switching from HbF to HbA.

SCD is the most common inherited hematologic disease in the United States, affecting approximately 80,000 people (Brousseau, 2010). SCD is most common in people of African ancestry, for whom the prevalence of SCD is 1 in 500. In Africa, the prevalence of SCD is 15 million. SCD is also more common in people of Indian, Saudi Arabian and Mediterranean descent. In those of Hispanic-American descent, the prevalence of sickle cell disease is 1 in 1,000.

SCD is caused by a single homozygous mutation in the HBB gene, c.17A>T (HbS mutation). The sickle mutation is a point mutation (GAG>GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less elastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood, the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, Hepatitis B, and Hepatitis C. Subjects may also suffer from acute chest crises and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-thalassemia is estimated to affect approximately 1 in 100,000 people worldwide. Its prevalence is higher in certain populations, including those of European descent, where its prevalence is approximately 1 in 10,000. β-thalassemia major, the more severe form of the disease, is life-threatening unless treated with lifelong blood transfusions and chelation therapy. In the United States, there are approximately 3,000 subjects with β-thalassemia major. β-thalassemia intermedia does not require blood transfusions, but it may cause growth delay and significant systemic abnormalities, and it frequently requires lifelong chelation therapy.

β-thalassemia is caused by mutations in the HBB gene. The most common HBB mutations leading to β-thalassemia are: c.−136C>G, c.92+1G>A, c.92+6T>C, c.93−21G>A, c.118C>T, c.316−106C>G, c.25_26delAA, c.27_28insG, c.92+5G>C, c.118C>T, c.135delC, c.315+1G>A, c.−78A>G, c.52A>T, c.59A>G, c.92+5G>C, c.124_127delTTCT, c.316−197C>T, c.−78A>G, c.52A>T, c.124_127delTTCT, c.316−197C>T, c.−138C>T, c.−79A>G, c.92+5G>C, c.75T>A, c.316−2A>G, and c.316−2A>C. These and other mutations associated with β-thalassemia cause mutated or absent β-globin chains, which causes a disruption of the normal Hb α-hemoglobin to β-hemoglobin ratio. Excess α-globin chains precipitate in erythroid precursors in the bone marrow.

In β-thalassemia major, both alleles of HBB contain nonsense, frameshift, or splicing mutations that leads to complete absence of β-globin production (denoted β⁰/β⁰). β-thalassemia major results in severe reduction in β-globin chains, leading to significant precipitation of α-globin chains in RBCs and more severe anemia.

β-thalassemia intermedia results from mutations in the 5′ or 3′ untranslated region of HBB, mutations in the promoter region or polyadenylation signal of HBB, or splicing mutations within the HBB gene. Patient genotypes are denoted βo/β+ or β+/β+. βo represents absent expression of a β-globin chain; (3+ represents a dysfunctional but present β-globin chain. Phenotypic expression varies among patients. Since there is some production of β-globin, β-thalassemia intermedia results in less precipitation of α-globin chains in the erythroid precursors and less severe anemia than β-thalassemia major. However, there are more significant consequences of erythroid lineage expansion secondary to chronic anemia.

Subjects with β-thalassemia major present between the ages of 6 months and 2 years, and suffer from failure to thrive, fevers, hepatosplenomegaly, and diarrhea. Adequate treatment includes regular transfusions. Therapy for β-thalassemia major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-thalassemia intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-thalassemia intermedia includes splenectomy, folic acid supplementation, hydroxyurea therapy, and radiotherapy for extramedullary masses. Chelation therapy is used in subjects who develop iron overload.

Life expectancy is often diminished in β-thalassemia patients. Subjects with β-thalassemia major who do not receive transfusion therapy generally die in their second or third decade. Subjects with β-thalassemia major who receive regular transfusions and adequate chelation therapy can live into their fifth decade and beyond. Cardiac failure secondary to iron toxicity is the leading cause of death in β-thalassemia major subjects due to iron toxicity.

A variety of new treatments are currently in development for SCD and β-Thal. Delivery of an anti-sickling HBB gene via gene therapy is currently being investigated in clinical trials. However, the long-term efficacy and safety of this approach is unknown. Transplantation with hematopoietic stem cells (HSCs) from an HLA-matched allogeneic stem cell donor has been demonstrated to cure SCD and β-Thal, but this procedure involves risks including those associated with ablation therapy, which is required to prepare the subject for transplant, increases risk of life-threatening opportunistic infections, and risk of graft vs. host disease after transplantation. In addition, matched allogeneic donors often cannot be identified. Reactivation of HBG (HBG1, HBG2) gene expression and induction of fetal hemoglobin (HbF) is an important therapeutic strategy for ameliorating the clinical symptoms and severity of SCD. Hydroxyurea is the only US FDA-approved drug with proven efficacy to induce HbF in SCD patients, yet serious complications have been associated with its use. Over the last three decades, numerous additional pharmacological agents that reactivate HBG transcription in vitro have been investigated, but few have proceeded to FDA approval, with the exception of arginine butyrate and decitabine; however, neither drug met the requirements for routine clinical use due to difficulties with oral delivery and inability to achieve therapeutic levels. Accordingly, there is a need for improved therapeutic compositions and methods for treatment of these hemoglobinopathies. There is a need to develop gene editing platforms with superior efficacy in genome editing for treatment of these hemoglobinopathies.

### SUMMARY

The disclosure provides a composition comprising: a) a first guide RNA (gRNA) and a first fusion protein or a first polynucleotide encoding the first fusion protein comprising: a mutant Cas9 (dCas9) polypeptide or an inactivated nuclease domain thereof and a Clo051 polypeptide or a nuclease domain thereof, configured to form a complex with the first gRNA, and b) a second gRNA and a second fusion protein or a second polynucleotide encoding the second fusion protein comprising: a dCas9 polypeptide or an inactivated nuclease domain thereof and a Clo051 polypeptide or a nuclease domain thereof, configured to form a complex with the second gRNA, wherein i) the first gRNA comprises a first targeting sequence comprising a nucleotide sequence selected from SEQ ID NOs: 1, 5, 7, 13, 15 or 17; and the second gRNA comprises a second targeting sequence comprising a nucleotide sequence of SEQ ID NO: 3, or ii) the first gRNA comprises a first targeting sequence comprising a nucleotide sequence selected from SEQ ID NOs: 7, 9 or 13; and the second gRNA comprises a second targeting sequence comprising a nucleotide sequence of SEQ ID NO: 11.

In some embodiments, the first gRNA comprises a first scaffold sequence and the second gRNA comprises a second scaffold sequence, wherein the first scaffold sequence and the second scaffold sequence comprises a nucleotide sequence selected from SEQ ID NO: 20 or 21.

In some embodiments, i) the first gRNA comprises a nucleotide sequence selected from SEQ ID NOs: 2, 6, 8, 16 or 18; and the second gRNA comprises a nucleotide sequence of SEQ ID NO: 4, or ii) the first gRNA comprises a nucleotide sequence selected from SEQ ID NOs: 10, 14 or 19; and the second gRNA comprises a nucleotide sequence of SEQ ID NO: 12.

In some embodiments, the first gRNA, the second gRNA or both the first gRNA and the second gRNA comprises one or more chemical modifications of a ribonucleotide, a ribonucleotide base, or a phosphodiester bond. In some embodiments, the one or more chemical modification comprises at least one chemically modified phosphodiester bond. In some embodiments, the at least one chemically modified phosphodiester bond is a phosphorothioate bond. In some embodiments, the composition comprises at least two phosphorothioate bonds at the 5′-terminus of the first gRNA, the second gRNA or both the first gRNA and the second gRNA. In some embodiments, the composition comprises a 2′ O-Me chemical modification at the 3′-terminus of the first gRNA, the second gRNA or both the first gRNA and the second gRNA.

In some embodiments, the dCas9 of the fusion protein and/or the second fusion protein is derived from a S. pyogenes Cas9 polypeptide or a S. aureus Cas9 polypeptide. In some embodiments, the C-terminus of the dCas9 or inactivated nuclease domain thereof, is joined to N-terminus of the Clo051 polypeptide or nuclease domain thereof via peptide linker sequence selected from GGGGS or SEQ ID NO: 23. In some embodiments, the first fusion protein comprises the amino acid sequence of SEQ ID NO: 39, 41, 42, or 43. In some embodiments, the second fusion protein comprises the amino acid sequence of SEQ ID NO: 39, 41, 42 or 43. In some embodiments, the first fusion protein and the second fusion protein are identical. In some embodiments, the first fusion protein and the second fusion protein are different.

In some embodiments, the first polynucleotide encoding the first fusion protein, the second polynucleotide encoding the second fusion protein or both the first polynucleotide and the second polynucleotide are an mRNA. In some embodiments, the mRNA comprises a 5′-cap.

The disclosure provides a composition comprising: i) a first gRNA comprising a nucleotide sequence of SEQ ID NO: 2, a second gRNA comprising a nucleotide sequence of SEQ ID NO: 4, a first polynucleotide sequence encoding a first fusion protein of SEQ ID NO: 39 and a second polynucleotide sequence encoding a second fusion protein of SEQ ID NO: 39; ii) a first gRNA comprising a nucleotide sequence of SEQ ID NO: 6, a second gRNA comprising a nucleotide sequence of SEQ ID NO: 4, a first polynucleotide sequence encoding a first fusion protein of SEQ ID NO: 39 and a second polynucleotide sequence encoding a second fusion protein of SEQ ID NO: 39; iii) a first gRNA comprising a nucleotide sequence of SEQ ID NO: 8, a second gRNA comprising a nucleotide sequence of SEQ ID NO: 4, a first polynucleotide sequence encoding a first fusion protein of SEQ ID NO: 41 and a second polynucleotide sequence encoding a second fusion protein of SEQ ID NO: 41; iv) a first gRNA comprising a nucleotide sequence of SEQ ID NO: 10, a second gRNA comprising a nucleotide sequence of SEQ ID NO: 12, a first polynucleotide sequence encoding a first fusion protein of SEQ ID NO: 39 and a second polynucleotide sequence encoding a second fusion protein of SEQ ID NO: 42; v) a first gRNA comprising a nucleotide sequence of SEQ ID NO: 14, a second gRNA comprising a nucleotide sequence of SEQ ID NO: 12, a first polynucleotide sequence encoding a first fusion protein of SEQ ID NO: 42 and a second polynucleotide sequence encoding a second fusion protein of SEQ ID NO: 42; vi) a first gRNA comprising a nucleotide sequence of SEQ ID NO: 19, a second gRNA comprising a nucleotide sequence of SEQ ID NO: 12, a first polynucleotide sequence encoding a first fusion protein of SEQ ID NO: 42 and a second polynucleotide sequence encoding a second fusion protein of SEQ ID NO: 42; vii) a first gRNA comprising a nucleotide sequence of SEQ ID NO: 16, a second gRNA comprising a nucleotide sequence of SEQ ID NO: 4, a first polynucleotide sequence encoding a first fusion protein of SEQ ID NO: 41 and a second polynucleotide sequence encoding a second fusion protein of SEQ ID NO: 41; or viii) a first gRNA comprising a nucleotide sequence of SEQ ID NO: 18, a second gRNA comprising a nucleotide sequence of SEQ ID NO: 4, a first polynucleotide sequence encoding a first fusion protein of SEQ ID NO: 41 and a second polynucleotide sequence encoding a second fusion protein of SEQ ID NO: 41.

In some embodiments, the composition is encapsulated in at least one lipid nanoparticle (LNP) comprising: about 4.75% of a compound of Formula (I) by moles,

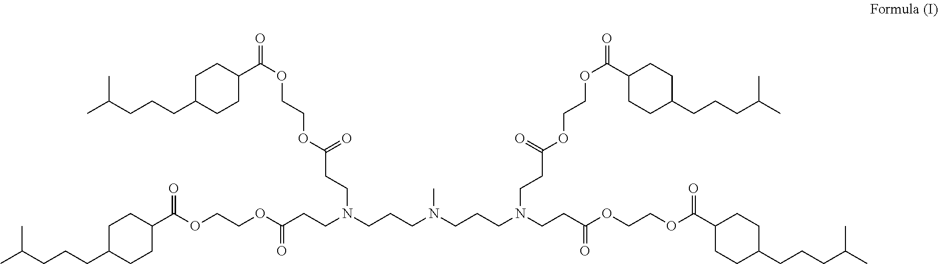 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

about 51.75% of cholesterol by moles, about 5% of DOPC by moles, and about 2.5% of DMG-PEG2000 by moles; wherein the first polynucleotide and the second polynucleotide is a RNA molecule, and wherein the ratio of lipid to RNA molecule in the at least one nanoparticle is about 120:1 (w/w).

In some embodiments, the composition is encapsulated in at least one LNP comprising: about 54% of SS-OP by moles, about 35% of cholesterol by moles, about 5% of DOPC by moles, about 5% of DSPC by moles, and about 1% of DMG-PEG2000 by moles, wherein the first polynucleotide and the second polynucleotide is a RNA molecule, and wherein the ratio of lipid to RNA molecule in the at least one nanoparticle is about 100:1 (w/w) and the total lipid of 25 nM.

In some embodiments, the composition is for use in modifying a HBG1 gene, a HBG2 gene, a BCL11A gene or a combination thereof in a cell.

The disclosure provides a method of modifying a population of cells comprising contacting the population of cells with any one of the compositions of the disclosure, wherein the first gRNA forms a complex with the first targeting sequence and the first fusion protein, and the second gRNA forms a complex with the second targeting sequence and the second fusion protein, thereby generating an indel between the first targeting sequence and the second targeting sequence and producing a modified population of cells. In some embodiments, the indel causes inactivation of a BCL11A gene.

In some embodiments, the modified population of cells have about 4-fold to about 9-fold increase in the expression of gamma globulin relative to an unmodified population of cells. In some embodiments, the modified population of cells have an increased level of fetal hemoglobin (HbF) expression relative to an unmodified population of cells.

In some embodiments, the cells are hematopoietic stem and precursor cells (HSPCs). In some embodiments, the HSPCs are capable of differentiating into erythroid progenitor cells.

The disclosure provides a population of cells modified according to any one of the methods of the disclosure.

The disclosure provides a method of treating a beta-hemoglobinopathy in a subject in need thereof, comprising administering to a subject, any one of the compositions or the cells of the disclosure. In some embodiments, the beta-hemoglobinopathy is beta-thalassemia or sickle cell disease.

### BRIEF DESCRIPTION OF THE DRAWINGS

FIG. 1 is a graph showing indels in HSPCs following editing with compositions comprising Cas-CLOVER and different concentrations of gRNA pairs targeting HBG1 or HBG2. On the y-axis, indels are shown as a percent modified reads out of total sequencing reads. The x-axis shows the gRNA pairs that were tested (Pair #1, Pair #2, Pair #3 and Pair #4). Each gRNA pair was tested at three increasing concentrations, 40, 200 and 400 μg/ml.

FIG. 2 is a graph showing indels in HSPCs following editing with compositions comprising Cas-CLOVER and either a high concentration (400 μg/ml) or a low concentration (200 μg/ml) of gRNA pairs targeting HBG1 or HBG2.

FIG. 3 is a graph showing indels in HSPCs following editing with compositions comprising Cas-CLOVER and different concentrations of gRNA pairs targeting HBG1 or HBG2. On the y-axis, indels are shown as a percent modified reads out of total sequencing reads. The x-axis shows the gRNA pairs that were tested (Pair #2, Pair #3 and Pair #4).

FIG. 4A-4B are a series of graphs showing the absolute number of Colony-forming unit (CFU) types for HSPCs following editing with compositions comprising Cas-CLOVER and different gRNA pairs targeting HBG1 or HBG2. Three controls were tested: EP only (nucleofection/electroporation only), CC only (Cas-CLOVER mRNA only) and sgRNA only. The following cell types were assayed:
CFU-GEMM (CFU-Granulocyte/Erythrocyte/Macrophage/Megakaryocyte),
CFU-GM (CFU-Granulocyte/Macrophage), BFU-E (Burst-forming Unit-Erythroid); and
CFU-E (CFU-Erythroid).

FIG. 5 is a graph showing HBG mRNA expression in HSPCs following editing with compositions comprising Cas-CLOVER and different gRNA pairs targeting HBG1 or HBG2. RT-qPCR results were normalized to HBB at Days 14, 18 and 21 during eryhroid differentiation. Adult and umbilical cord blood samples were used as negative and positive controls, respectively.

FIG. 6 is a graph showing HbF protein expression levels in HSPCs following editing with compositions comprising Cas-CLOVER and different gRNA pairs targeting HBG1 or HBG2. The left y-axis shows the percentage of F-cells determined by flow cytometry. The right y-axis shows mean fluorescence intensity of HbF signal per F-cell relative to the EP only (nucleofection/electroporation only) control.

FIG. 7 is a schematic diagram of the composition of the disclosure. A first fusion protein (e.g. Cas-Clover comprising a dCas9-linker-Clo051) is complexed with a first gRNA at the 5′ terminus of the genomic region. A second fusion protein (e.g. Cas-Clover comprising a dCas9-linker Clo051) is complexed with a second gRNA at the 3′ terminus of the genomic region. Targeting using gRNAs provides highly efficient and accurate targeting. Only when the Clo051 nucleases of the first fusion protein and the second fusion protein are brought in proximity, is a cut made to the genomic DNA template. In some instances, the cut a HBG1 gene, a HBG2 gene, a BCL11A gene or a combination thereof.

FIG. 8 is a graph showing indels in HSPCs following editing with compositions comprising Cas-CLOVER variants and gRNA pairs targeting HBG1 or HBG2.

All documents cited herein, including any cross referenced or related patent or application is hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### DETAILED DESCRIPTION

The present disclosure provides compositions and methods for genetically modifying a genome to include a polynucleotide insertion, deletion and/or a substitution into chromosomal DNA that enhance the transcription of HBG1 and/or HBG2 genes, which encode the γA and γG subunits of hemoglobin, respectively. In particular, the present disclosure overcomes problems associated with current technologies by providing a method for efficiently genetically modifying cellular genomes to include polynucleotide insertions, deletions and/or substitutions, which is advantageous in therapeutic expression of fetal hemoglobin (HbF) for treatment of hemoglobinopathies.

Fetal hemoglobin (HbF) expression can be induced using various genome strategies. For example, HbF expression can be induced through targeted disruption of the region proximal to the HBG1 and HBG2 promoter target sequence, and or the erythroid cell specific expression of a transcriptional repressor, BCL11A (also discussed in commonly-assigned International Patent Publication No. WO 2015/148860 by Friedland et al. (“Friedland”), published Oct. 1, 2015, which is incorporated by reference in its entirety herein), which encodes a repressor that silences HBG1 and HBG2 (Canvers 2015). Genetic mapping and genome-wide association studies discovered genetic loci in B-cell lymphoma/leukemia 11A (BCL11A), an Xnm1 variant upstream of hemoglobin subunit gamma 1 (HBG1). Supporting studies reported that hemoglobin switching is controlled through the activity of epigenetic modulators such as BCL11A. The HBG promoter region for both HBG1 and HBG2 genes comprises a DNA binding region for BCL11A, which is a potent silencer of HbF expression. Binding of BCL11A to the HBGB promoter inhibits expression of gamma subunits 1 and 2, can lead to inhibition of the γ- to β-globin switching process.

The genome editing systems of this disclosure can include two or more fusion proteins (e.g., Cas-Clover) and two or more gRNAs having a targeting domain that is complementary to a sequence in or near the target region. In certain embodiments, the DNA binding region of BCL11A is targeted for disruption. In certain embodiments, the promoter region of HBG1 and/or HBG2 is targeted for disruption. In certain embodiments, genome editing systems disclosed herein may be used to introduce a polynucleotide insertion, deletion and/or a substitution in the targeted region.

The treatment of hemoglobinopathies by gene therapy and/or genome editing is complicated by the fact that the cells that are phenotypically affected by the disease, erythrocytes or RBCs, are enucleated, and do not contain genetic material encoding either the aberrant hemoglobin protein (Hb) subunits nor the γA or γG subunits targeted in the exemplary genome editing approaches described above. This complication is addressed, in certain embodiments of this disclosure, by the alteration of cells that are competent to differentiate into, or otherwise give rise to, erythrocytes. Cells within the erythroid lineage that are altered according to various embodiments of this disclosure include, without limitation, hematopoietic stem and progenitor cells (HSPCs), erythroblasts (including basophilic, polychromatic and/or orthochromatic erythroblasts), proerythroblasts, polychromatic erythrocytes or reticulocytes, embryonic stem (ES) cells, and/or induced pluripotent stem (iPSC) cells. These cells may be altered in situ (e.g., within a tissue of a subject) or ex vivo.

The present disclosure overcomes problems associated with current technologies by providing compositions comprising genetically engineered fusion molecules (e.g. Cas-Clover) for targeted reduction or elimination of gene products in a cell for use in in vivo gene therapy. The compositions comprising genetically engineered fusion molecules of the disclosure are useful for treatment of genetic diseases. Non-limiting examples of genetic diseases include, hemoglobinopathies such as sickle cell disease or beta thalassemia. Accordingly, methods of making genetically engineered fusion molecules and pharmaceutical formulations thereof (e.g., lipid nanoparticle formulations) for use in in vivo delivery are also provided. As a non-limiting example, the magnitude of the improvement provided by the compositions the disclosure, could cross a key therapeutic threshold to fully enable activation of fetal hemoglobin (HbF) expression, which would provide therapeutic efficacy for functional correction of sickle cell disease, or beta thalassemia.

### Methods for Targeted Genome Editing at Selected Locus

### Gene Editing Compositions and Methods

The present disclosure provides a gene editing composition a well as a cell comprising the gene editing composition. The gene editing composition can comprise a sequence encoding a DNA localization domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The DNA localization domain can comprise one or more of a CRISPR/Cas protein, a Transcription Activator-Like Effector Nuclease (TALEN), a Zinc Finger Nuclease (ZFN), and an endonuclease.

Exemplary dCas9-Clo051 (Cas-CLOVER) Fusion Proteins

The nuclease protein or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. The gene editing composition can further comprise a guide sequence. In some embodiments, the guide sequence comprises an RNA sequence.

The disclosure provides compositions comprising a Cas9 operatively linked to an effector. The disclosure provides a fusion protein comprising, consisting essentially of or consisting of a DNA localization component and an effector molecule, wherein the effector comprises a Cas9. A Cas9 construct of the disclosure can comprise an effector comprising a type IIS endonuclease. A Staphylococcus aureus Cas9 with an active catalytic site comprises the amino acid sequence of SEQ ID NO: 30.

The disclosure provides compositions comprising an inactivated, Cas9 (dSaCas9) operatively linked to an effector. The disclosure provides a fusion protein comprising, consisting essentially of or consisting of a DNA localization component and an effector molecule, wherein the effector comprises a inactivated Cas9 (dSaCas9). An inactivated Cas9 (dSaCas9) construct of the disclosure can comprise an effector comprising a type IIS endonuclease. A dSaCas9 comprises the amino acid sequence of SEQ ID NO: 31, which includes a D10A and a N580A mutation to inactivate the catalytic site.

The disclosure provides compositions comprising an inactivated Cas9 (dCas9) operatively linked to an effector. The disclosure provides a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptococcus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 32 or SEQ ID NO: 33.

An exemplary Clo051 nuclease domain comprises, consists essentially of or consists of, the amino acid sequence of SEQ ID NO: 34. In some aspects, the Clo051 nuclease domain comprises at least one amino acid substitution. In some aspects, the amino acid substitution is in the alpha-helix-loop domain of the Clo051 nuclease. In some aspects, the amino acid substitution is at position 35, 37, 60, 92, 98, 100 or 146 of SEQ ID NO: 34. In some aspects, the amino acid substitution is at position 37 of SEQ ID NO: 34. In some aspects, the amino acid substitution is at positions 37 and 92 of SEQ ID NO: 34.

An exemplary dCas9-Clo051 (Cas-CLOVER) fusion protein can comprise, consist essentially of, or consist of, the amino acid sequence of SEQ ID NO: 35. The exemplary dCas9-Clo051 fusion protein can be encoded by a polynucleotide which comprises, consists essentially of, or consists of, the nucleic acid sequence of SEQ ID NO: 36. The nucleic acid encoding the dCas9-Clo051 fusion protein can be DNA or RNA.

An exemplary dCas9-Clo051 (Cas-CLOVER) fusion protein can comprise, consist essentially of, or consist of, the amino acid sequence of SEQ ID NO: 37. The exemplary dCas9-Clo051 fusion protein can be encoded by a polynucleotide which comprises, consists essentially of, or consists of, the nucleic acid sequence of SEQ ID NO: 38. The nucleic acid encoding the dCas9-Clo051 fusion protein can be DNA or RNA.

An exemplary dCas9-Clo051 fusion (Cas-CLOVER) fusion protein of the disclosure may further comprise at least one nuclear localization sequence (NLS). In some embodiments, the dCas9-Clo051 fusion protein of the disclosure comprises at least two nuclear localization sequences. In some embodiments, the NLS is on the N′terminal end of the dCas9-Clo051 fusion protein (NLS-dCas9-Clo051). In some embodiments, the NLS is on the C-terminal end of the dCas9-Clo051 fusion protein (dCas9-Clo051-NLS). In some embodiments, the NLS is on the N′terminal end and at the C′terminal end of the dCas9-Clo051 fusion protein (“NLS-dCas9-Clo051-NLS” or “wildtype Cas-CLOVER” or “dspCas9 Ca-CLOVER”).

The NLS-dCas9-Clo051-NLS (“wildtype Cas-CLOVER” or “dspCas9 Cas-CLOVER”) fusion protein can comprise, consist essentially of, or consist of, the amino acid sequence of SEQ ID NO: 39.

dspCas9 Cas-CLOVER amino acid sequence (NLS amino acid sequence is bolded and underlined; linker is bolded and italicized)

(SEQ ID NO: 39)
MAPKKKRKVEGIKSNISLLKDELRGQISHISHEYLSLIDLAFDSKQ
NRLFEMKVLELLVNEYGFKGRHLGGSRKPDGIVYSTTLEDNFGIIV
DTKAYSEGYSLPISQADEMERYVRENSNRDEEVNPNKWWENFSEEV
KKYYFVFISGSFKGKFEEQLRRLSMTTGVNGSAVNVVNLLLGAEKI
RSGEMTIEELERAMFNNSEFILKYGGGGSDKKYSIGLAIGTNSVGW
AVITDEYKVPSKKFKVLGNTDRHSIKKNLIGALLFDSGETAEATRL
KRTARRRYTRRKNRICYLQEIFSNEMAKVDDSFFHRLEESFLVEED
KKHERHPIFGNIVDEVAYHEKYPTIYHLRKKLVDSTDKADLRLIYL
ALAHMIKFRGHFLIEGDLNPDNSDVDKLFIQLVQTYNQLFEENPIN
ASGVDAKAILSARLSKSRRLENLIAQLPGEKKNGLFGNLIALSLGL
TPNFKSNFDLAEDAKLQLSKDTYDDDLDNLLAQIGDQYADLFLAAK
NLSDAILLSDILRVNTEITKAPLSASMIKRYDEHHQDLTLLKALVR
QQLPEKYKEIFFDQSKNGYAGYIDGGASQEEFYKFIKPILEKMDGT
EELLVKLNREDLLRKQRTFDNGSIPHQIHLGELHAILRRQEDFYPF
LKDNREKIEKILTFRIPYYVGPLARGNSRFAWMTRKSEETITPWNF
EEVVDKGASAQSFIERMTNFDKNLPNEKVLPKHSLLYEYFTVYNEL
TKVKYVTEGMRKPAFLSGEQKKAIVDLLFKTNRKVTVKQLKEDYFK
KIECFDSVEISGVEDRFNASLGTYHDLLKIIKDKDFLDNEENEDIL
EDIVLTLTLFEDREMIEERLKTYAHLFDDKVMKQLKRRRYTGWGRL
SRKLINGIRDKQSGKTILDFLKSDGFANRNFMQLIHDDSLTFKEDI
QKAQVSGQGDSLHEHIANLAGSPAIKKGILQTVKVVDELVKVMGRH
KPENIVIEMARENQTTQKGQKNSRERMKRIEEGIKELGSQILKEHP
VENTQLQNEKLYLYYLQNGRDMYVDQELDINRLSDYDVDAIVPQSF
LKDDSIDNKVLTRSDKNRGKSDNVPSEEVVKKMKNYWRQLLNAKLI
TQRKFDNLTKAERGGLSELDKAGFIKRQLVETRQITKHVAQILDSR
MNTKYDENDKLIREVKVITLKSKLVSDFRKDFQFYKVREINNYHHA
HDAYLNAVVGTALIKKYPKLESEFVYGDYKVYDVRKMIAKSEQEIG
KATAKYFFYSNIMNFFKTEITLANGEIRKRPLIETNGETGEIVWDK
GRDFATVRKVLSMPQVNIVKKTEVQTGGFSKESILPKRNSDKLIAR
KKDWDPKKYGGFDSPTVAYSVLVVAKVEKGKSKKLKSVKELLGITI
MERSSFEKNPIDFLEAKGYKEVKKDLIIKLPKYSLFELENGRKRML
ASAGELQKGNELALPSKYVNFLYLASHYEKLKGSPEDNEQKQLFVE
QHKHYLDEIIEQISEFSKRVILADANLDKVLSAYNKHRDKPIREQA
ENIIHLFTLTNLGAPAAFKYFDTTIDRKRYTSTKEVLDATLIHQSI
TGLYETRIDLSQLGGDGSPKKKRKVSS.

The nucleic acid encoding the NLS-dCas9-Clo051-NLS (“wildtype Cas-CLOVER” or “dspCas9 Cas-CLOVER”) fusion protein can be DNA or RNA. In some embodiments, a dCas9-Clo051 fusion protein comprising two NLS regions is encoded by an mRNA sequence comprising, consisting essentially of or consisting of SEQ ID NO: 40.

The NLS-dCas9-Clo051-NLS (“dspCas9-XL Cas-CLOVER” or “dspCas9 5xGGGGS Cas-CLOVER”) fusion protein can comprise, consist essentially of, or consist of, the amino acid sequence of SEQ ID NO: 41.

“dspCas9-XL Cas-CLOVER” amino acid sequence (NLS amino acid sequence is bolded and underlined; linker is bolded and italicized)

(SEQ ID NO: 41)
MAPKKKRKVEGIKSNISLLKDELRGQISHISHEYLSLIDLAFDSKQ
NRLFEMKVLELLVNEYGFKGRHLGGSRKPDGIVYSTTLEDNFGIIV
DTKAYSEGYSLPISQADEMERYVRENSNRDEEVNPNKWWENFSEEV
KKYYFVFISGSFKGKFEEQLRRLSMTTGVNGSAVNVVNLLLGAEKI
RSGEMTIEELERAMFNNSEFILKYGGGGSGGGGSGGGGSGGGGSGG
GGSDKKYSIGLAIGTNSVGWAVITDEYKVPSKKFKVLGNTDRHSIK
KNLIGALLFDSGETAEATRLKRTARRRYTRRKNRICYLQEIFSNEM
AKVDDSFFHRLEESFLVEEDKKHERHPIFGNIVDEVAYHEKYPTIY
HLRKKLVDSTDKADLRLIYLALAHMIKFRGHFLIEGDLNPDNSDVD
KLFIQLVQTYNQLFEENPINASGVDAKAILSARLSKSRRLENLIAQ
LPGEKKNGLFGNLIALSLGLTPNFKSNFDLAEDAKLQLSKDTYDDD
LDNLLAQIGDQYADLFLAAKNLSDAILLSDILRVNTEITKAPLSAS
MIKRYDEHHQDLTLLKALVRQQLPEKYKEIFFDQSKNGYAGYIDGG
ASQEEFYKFIKPILEKMDGTEELLVKLNREDLLRKQRTFDNGSIPH
QIHLGELHAILRRQEDFYPFLKDNREKIEKILTFRIPYYVGPLARG
NSRFAWMTRKSEETITPWNFEEVVDKGASAQSFIERMTNFDKNLPN
EKVLPKHSLLYEYFTVYNELTKVKYVTEGMRKPAFLSGEQKKAIVD
LLFKTNRKVTVKQLKEDYFKKIECFDSVEISGVEDRFNASLGTYHD
LLKIIKDKDFLDNEENEDILEDIVLTLTLFEDREMIEERLKTYAHL
FDDKVMKQLKRRRYTGWGRLSRKLINGIRDKQSGKTILDFLKSDGF
ANRNFMQLIHDDSLTFKEDIQKAQVSGQGDSLHEHIANLAGSPAIK
KGILQTVKVVDELVKVMGRHKPENIVIEMARENQTTQKGQKNSRER
MKRIEEGIKELGSQILKEHPVENTQLQNEKLYLYYLQNGRDMYVDQ
ELDINRLSDYDVDAIVPQSFLKDDSIDNKVLTRSDKNRGKSDNVPS
EEVVKKMKNYWRQLLNAKLITQRKFDNLTKAERGGLSELDKAGFIK
RQLVETRQITKHVAQILDSRMNTKYDENDKLIREVKVITLKSKLVS
DFRKDFQFYKVREINNYHHAHDAYLNAVVGTALIKKYPKLESEFVY
GDYKVYDVRKMIAKSEQEIGKATAKYFFYSNIMNFFKTEITLANGE
IRKRPLIETNGETGEIVWDKGRDFATVRKVLSMPQVNIVKKTEVQT
GGFSKESILPKRNSDKLIARKKDWDPKKYGGFDSPTVAYSVLVVAK
VEKGKSKKLKSVKELLGITIMERSSFEKNPIDFLEAKGYKEVKKDL
IIKLPKYSLFELENGRKRMLASAGELQKGNELALPSKYVNFLYLAS
HYEKLKGSPEDNEQKQLFVEQHKHYLDEIIEQISEFSKRVILADAN
LDKVLSAYNKHRDKPIREQAENIIHLFTLTNLGAPAAFKYEDTTID
RKRYTSTKEVLDATLIHQSITGLYETRIDLSQLGGDGSPKKKRKVS
S

The NLS-dCas9-Clo051-NLS (“dsaCas9 Cas-CLOVER”) fusion protein can comprise, consist essentially of, or consist of, the amino acid sequence of SEQ ID NO: 42.

“dsaCas9 Cas-CLOVER” amino acid sequence (NLS amino acid sequence is bolded and underlined; linker is bolded and italicized

(SEQ ID NO: 42)
MAPKKKRKVEGIKSNISLLKDELRGQISHISHEYLSLIDLAFDSKQ
NRLFEMKVLELLVNEYGFKGRHLGGSRKPDGIVYSTTLEDNFGIIV
DTKAYSEGYSLPISQADEMERYVRENSNRDEEVNPNKWWENFSEEV
KKYYFVFISGSFKGKFEEQLRRLSMTTGVNGSAVNVVNLLLGAEKI
RSGEMTIEELERAMFNNSEFILKYGGGGSKRNYILGLAIGITSVGY
GIIDYETRDVIDAGVRLFKEANVENNEGRRSKRGARRLKRRRRHRI
QRVKKLLFDYNLLTDHSELSGINPYEARVKGLSQKLSEEEFSAALL
HLAKRRGVHNVNEVEEDTGNELSTKEQISRNSKALEEKYVAELQLE
RLKKDGEVRGSINRFKTSDYVKEAKQLLKVQKAYHQLDQSFIDTYI
DLLETRRTYYEGPGEGSPFGWKDIKEWYEMLMGHCTYFPEELRSVK
YAYNADLYNALNDLNNLVITRDENEKLEYYEKFQIIENVFKQKKKP
TLKQIAKEILVNEEDIKGYRVTSTGKPEFTNLKVYHDIKDITARKE
IIENAELLDQIAKILTIYQSSEDIQEELTNLNSELTQEEIEQISNL
KGYTGTHNLSLKAINLILDELWHTNDNQIAIFNRLKLVPKKVDLSQ
QKEIPTTLVDDFILSPVVKRSFIQSIKVINAIIKKYGLPNDIIIEL
AREKNSKDAQKMINEMQKRNRQTNERIEEIIRTTGKENAKYLIEKI
KLHDMQEGKCLYSLEAIPLEDLLNNPFNYEVDHIIPRSVSFDNSFN
NKVLVKQEEASKKGNRTPFQYLSSSDSKISYETFKKHILNLAKGKG
RISKIKKEYLLEERDINRFSVQKDFINRNLVDTRYATRGLMNLLRS
YFRVNNLDVKVKSINGGFTSFLRRKWKFKKERNKGYKHHAEDALII
ANADFIFKEWKKLDKAKKVMENQMFEEKQAESMPEIETEQEYKEIF
ITPHQIKHIKDFKDYKYSHRVDKKPNRELINDTLYSTRKDDKGNTL
IVNNLNGLYDKDNDKLKKLINKSPEKLLMYHHDPQTYQKLKLIMEQ
YGDEKNPLYKYYEETGNYLTKYSKKDNGPVIKKIKYYGNKLNAHLD
ITDDYPNSRNKVVKLSLKPYRFDVYLDNGVYKFVTVKNLDVIKKEN
YYEVNSKCYEEAKKLKKISNQAEFIASFYNNDLIKINGELYRVIGV
NNDLLNRIEVNMIDITYREYLENMNDKRPPRIIKTIASKTQSIKKY
STDILGNLYEVKSKKHPQIIKKGGSPKKKRKVSS

The NLS-dCas9-Clo051-NLS (“dsaCas9-XL Cas-CLOVER” of dsaCas9 5xGGGGS Cas-CLOVER”) fusion protein can comprise, consist essentially of, or consist of, the amino acid sequence of SEQ ID NO: 43.

“dsaCas9-XL Cas-CLOVER” amino acid sequence (NLS amino acid sequence is bolded and underlined; linker is bolded and italicized)

(SEQ ID NO: 43)
MAPKKKRKVEGIKSNISLLKDELRGQISHISHEYLSLIDLAFDSKQ
NRLFEMKVLELLVNEYGFKGRHLGGSRKPDGIVYSTTLEDNFGIIV
DTKAYSEGYSLPISQADEMERYVRENSNRDEEVNPNKWWENFSEEV
KKYYFVFISGSFKGKFEEQLRRLSMTTGVNGSAVNVVNLLLGAEKI
RSGEMTIEELERAMFNNSEFILKYGGGGSGGGGSGGGGSGGGGSGG
GGSKRNYILGLAIGITSVGYGIIDYETRDVIDAGVRLFKEANVENN
EGRRSKRGARRLKRRRRHRIQRVKKLLFDYNLLTDHSELSGINPYE
ARVKGLSQKLSEEEFSAALLHLAKRRGVHNVNEVEEDTGNELSTKE
QISRNSKALEEKYVAELQLERLKKDGEVRGSINRFKTSDYVKEAKQ
LLKVQKAYHQLDQSFIDTYIDLLETRRTYYEGPGEGSPFGWKDIKE
WYEMLMGHCTYFPEELRSVKYAYNADLYNALNDLNNLVITRDENEK
LEYYEKFQIIENVFKQKKKPTLKQIAKEILVNEEDIKGYRVTSTGK
PEFTNLKVYHDIKDITARKEIIENAELLDQIAKILTIYQSSEDIQE
ELTNLNSELTQEEIEQISNLKGYTGTHNLSLKAINLILDELWHTND
NQIAIFNRLKLVPKKVDLSQQKEIPTTLVDDFILSPVVKRSFIQSI
KVINAIIKKYGLPNDIIIELAREKNSKDAQKMINEMQKRNRQTNER
IEEIIRTTGKENAKYLIEKIKLHDMQEGKCLYSLEAIPLEDLLNNP
FNYEVDHIIPRSVSFDNSFNNKVLVKQEEASKKGNRTPFQYLSSSD
SKISYETFKKHILNLAKGKGRISKTKKEYLLEERDINRFSVQKDFI
NRNLVDTRYATRGLMNLLRSYFRVNNLDVKVKSINGGFTSFLRRKW
KFKKERNKGYKHHAEDALIIANADFIFKEWKKLDKAKKVMENQMFE
EKQAESMPEIETEQEYKEIFITPHQIKHIKDFKDYKYSHRVDKKPN
RELINDTLYSTRKDDKGNTLIVNNLNGLYDKDNDKLKKLINKSPEK
LLMYHHDPQTYQKLKLIMEQYGDEKNPLYKYYEETGNYLTKYSKKD
NGPVIKKIKYYGNKLNAHLDITDDYPNSRNKVVKLSLKPYRFDVYL
DNGVYKFVTVKNLDVIKKENYYEVNSKCYEEAKKLKKISNQAEFIA
SFYNNDLIKINGELYRVIGVNNDLLNRIEVNMIDITYREYLENMND
KRPPRIIKTIASKTQSIKKYSTDILGNLYEVKSKKHPQIIKKGGSP
KKKRKVSS

A cell comprising the gene editing composition can express the gene editing composition stably or transiently.

gRNAs

As used herein, the term “guide sequence” or “spacer” in the context of a Cas-Clover system or a CRISPR-Cas9 system, comprises any polynucleotide molecule having sufficient complementarity with a target nucleic acid sequence to hybridize with the target nucleic acid sequence and direct sequence-specific binding of a nucleic acid-targeting complex to the target nucleic acid sequence. The guide sequence may comprise both RNA and DNA polynucleotides. The guide sequence may form a duplex with a target sequence. The duplex may be a DNA duplex, an RNA duplex, or a RNA/DNA duplex. The terms “guide molecule”, “guide RNA”, “gRNA”, “single guide RNA” and “sgRNA” are used interchangeably herein to refer to RNA-based molecules that are capable of forming a complex with a Cas-Clover or a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA may encompass RNA-based molecules having one or more chemically modifications (e.g., by chemical linking two ribonucleotides or by replacement of one or more ribonucleotides with one or more deoxyribonucleotides), as described herein. The guide sequence may also partially comprise RNA and DNA-based nucleotides in which the molecule is chimeric for RNA and DNA nucleobases (e.g., containing either ribose or deoxyribose sugars).

The term “target region”, “target sequence” or “protospacer” as used interchangeably herein refers to the region of the target gene or genomic target site, to which the Cas-Clover system or the CRISPR/Cas9-based system targets. The Cas-Clover or the CRISPR/Cas9-based system may include at least one gRNA, wherein the gRNAs target different DNA sequences. The target DNA sequences may be overlapping. The Cas-Clover system may include at least two gRNAs, wherein the gRNAs target different DNA sequences. The target sequence or protospacer may be followed by a PAM sequence at the 3′ end of the protospacer. Different Type II CRISPR systems have differing PAM requirements. For example, the S. pyogenes Type II system uses an “NGG” sequence, where “N” can be any nucleotide.

The guide RNA or the guide RNA of a Cas-Clover protein or a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the Cas-Clover or the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In some embodiments, the guide RNA comprises a guide sequence and a scaffold sequence. In some embodiments, the scaffold sequence is isolated from Streptococcus pyogenes. In some embodiments, the Streptococcus pyogenes scaffold sequence comprises the nucleic acid sequence:

GUUUUAGAGCUAGAAAUAGCAAGUUAAAAUAAGGCUAGUCCGUUAUCAACUUGA AAAAGUGGCACCGAGUCGGUGCUUUU (SEQ ID NO: 19). In some embodiments, the scaffold sequence is isolated from Staphylococcus aureus. In some embodiments, the Staphylococcus aureus scaffold sequence comprises the nucleic acid sequence:

(SEQ ID NO: 20)
GUUUUAGUACUCUGGAAACAGAAUCUACUAAAACAAGGCAAAAUGC
CGUGUUUAUCUCGUCAACUUGUUGGCGAGAUUUU.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 nucleotides in length.

In some embodiments, the sequence of the guide molecule (direct repeat and/or spacer) is selected to reduce the degree of secondary structure within the guide molecule. In some embodiments, about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

As described above, the Cas-Clover system and the CRISPR/Cas9 system utilizes one or more targeting gRNAs that provides the targeting of the Cas-Clover system and the CRISPR/Cas9-based system. The gRNA may be a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

In some embodiments, the gRNA targets a BCL11A binding site in a HBG promoter region upstream of the target gene (e.g., HBG1 or HBG2 gene locus), e.g., between 0-1000 bp upstream of a target gene). In some embodiments, the gRNA targets near a BCL11A binding site in a HBG promoter region upstream of HBG1 gene locus or HBG2 gene locus. In some embodiments the gRNA targets a region between 0-50 bp, 0-100 bp, 0-150 bp, 0-200 bp, or 0-250 bp upstream or downstream of the BCL11A binding site. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the target gene.

A gRNA can be divided into a target binding region and a Cas9 binding region. The target binding region hybridizes with a target region in a target gene or intergenic region. Methods for designing such target binding regions are known in the art, see, e.g., Doench et al., Nat Biotechnol. (2014) 32: 1262-7; and Doench et al., Nat Biotechnol. (2016) 34: 184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

Exemplary gRNAs of the disclosure include but are not limited to sequences for targeting HBG1 gene locus, HBG2 gene locus or the BCL11A binding site in a HBG promoter region of the HBG1 or HBG2 gene loci.

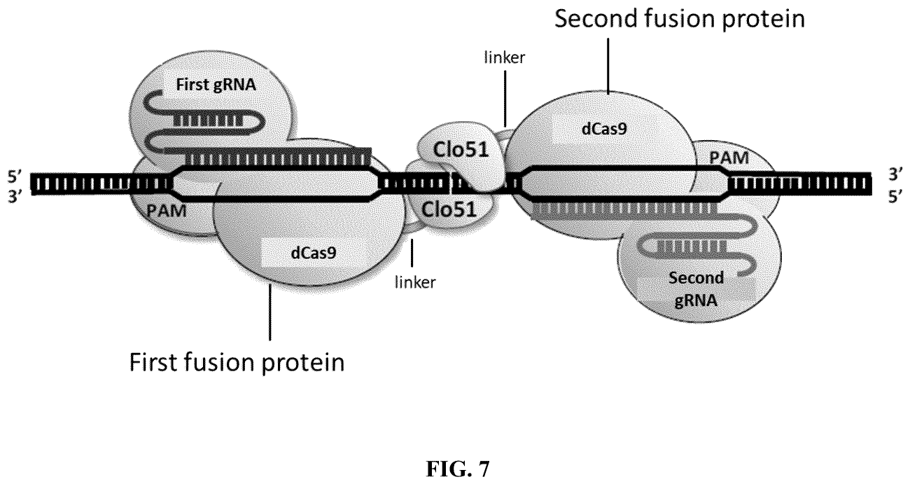

In some embodiments, the first gRNA (also referred to as “left gRNA”) binds to a template sequence at the 5′ terminus of the target gene locus and the second gRNA (also referred to as “right gRNA”) binds to a template sequence at the 3′ terminus of the target gene locus. A schematic diagram is shown in FIG. 7.

Exemplary gRNAs of the disclosure comprise, consist essentially of or consists of the target sequences and full length sequences as shown in Table 1.

TABLE 1
Exemplary gRNAs of the disclosure

| | Target/Full | | | |
|---|---|---|---|---|
| | Length | | | |
| Exemplary | gRNA | | | SEQ |
| gRNA | Sequence | Scaffold | Sequence | ID NO: |
| HBG 1/2- | Target | — | GCUAUUGGUCAAGGCAAGGC | SEQ ID |
| first/left gRNA | Sequence | | | NO: 1 |
| (Pair #1) | | | | |
| HBG 1/2- | gRNA | S. pyogenes | GCUAUUGGUCAAGGCAAGGCGUU | SEQ ID |
| first/left gRNA- | Sequence | | UUAGAGCUAGAAAUAGCAAGUUA | NO: 2 |
| (Pair #1) | | | AAAUAAGGCUAGUCCGUUAUCAA | |
| | | | CUUGAAAAAGUGGCACCGAGUCG | |
| | | | GUGCUUUU | |
| HBG 1/2- | Target | — | UAGUCUUAGAGUAUCCAGUG | SEQ ID |
| second/right | Sequence | | | NO: 3 |
| gRNA (Pair | | | | |
| #1, 2, 3, 7, 8) | | | | |
| HBG 1/2- | gRNA | S. pyogenes | UAGUCUUAGAGUAUCCAGUGGUU | SEQ ID |
| second/right | Sequence | | UUAGAGCUAGAAAUAGCAAGUUA | NO: 4 |
| gRNA (Pair | | | AAAUAAGGCUAGUCCGUUAUCAA | |
| #1, 2, 3, 7, 8) | | | CUUGAAAAAGUGGCACCGAGUCG | |
| | | | GUGCUUUU | |
| HBG 1/2- | Target | — | CAAGGCUAUUGGUCAAGGCA | SEQ ID |
| first/left gRNA- | Sequence | | | NO: 5 |
| (Pair #2) | | | | |
| HBG 1/2- | gRNA | S. pyogenes | CAAGGCUAUUGGUCAAGGCAGUU | SEQ ID |
| first/left gRNA- | Sequence | | UUAGAGCUAGAAAUAGCAAGUUA | NO: 6 |
| (Pair #2) | | | AAAUAAGGCUAGUCCGUUAUCAA | |
| | | | CUUGAAAAAGUGGCACCGAGUCG | |
| | | | GUGCUUUU | |
| HBG 1/2- | Target | — | AAGGCUGGCCAACCCAUGGG | SEQ ID |
| first/left gRNA- | Sequence | | | NO: 7 |
| (Pair #3, 6) | | | | |
| HBG 1/2- | gRNA | S. pyogenes | AAGGCUGGCCAACCCAUGGGGUU | SEQ ID |
| first/left gRNA | Sequence | | UUAGAGCUAGAAAUAGCAAGUUA | NO: 8 |
| (Pair #3) | | | AAAUAAGGCUAGUCCGUUAUCAA | |
| | | | CUUGAAAAAGUGGCACCGAGUCG | |
| | | | GUGCUUUU | |
| HBG 1/2- | Target | — | GGCAAGGCUGGCCAACCCAU | SEQ ID |
| first/left gRNA- | Sequence | | | NO: 9 |
| (Pair #4) | | | | |
| HBG 1/2- | gRNA | S. pyogenes | GGCAAGGCUGGCCAACCCAUGUU | SEQ ID |
| first/left gRNA- | Sequence | | UUAGAGCUAGAAAUAGCAAGUUA | NO: 10 |
| (Pair #4) | | | AAAUAAGGCUAGUCCGUUAUCAA | |
| | | | CUUGAAAAAGUGGCACCGAGUCG | |
| | | | GUGCUUUU | |
| HBG 1/2- | Target | — | GCAAACUUGACCAAUAGUCU | SEQ ID |
| second/right | Sequence | | | NO: 11 |
| gRNA (Pair | | | | |
| #4, 5, 6) | | | | |
| HBG 1/2- | gRNA | S. aureus | GCAAACUUGACCAAUAGUCUGUU | SEQ ID |
| second/right | Sequence | | UUAGUACUCUGGAAACAGAAUCU | NO: 12 |
| gRNA (Pair | | | ACUAAAACAAGGCAAAAUGCCGU | |
| #4, 5, 6) | | | GUUUAUCUCGUCAACUUGUUGGC | |
| | | | GAGAUUUU | |
| HBG 1/2- | Target | — | AAGGCAAGGCUGGCCAACCC | SEQ ID |
| first/left gRNA- | Sequence | | | NO: 13 |
| (Pair #5) | | | | |
| HBG 1/2- | gRNA | S. aureus | AAGGCAAGGCUGGCCAACCCGUU | SEQ ID |
| first/left gRNA- | Sequence | | UUAGUACUCUGGAAACAGAAUCU | NO: 14 |
| (Pair #5) | | | ACUAAAACAAGGCAAAAUGCCGU | |
| | | | GUUUAUCUCGUCAACUUGUUGGC | |
| | | | GAGAUUUU | |
| HBG 1/2- | Target | — | CCCAUGGGUGGAGUUUAGCC | SEQ ID |
| first/left gRNA- | Sequence | | | NO: 15 |
| (Pair #7) | | | | |
| HBG 1/2- | gRNA | S. pyogenes | CCCAUGGGUGGAGUUUAGCCGUU | SEQ ID |
| first/left gRNA- | Sequence | | UUAGAGCUAGAAAUAGCAAGUUA | NO: 16 |
| (Pair #7) | | | AAAUAAGGCUAGUCCGUUAUCAA | |
| | | | CUUGAAAAAGUGGCACCGAGUCG | |
| | | | GUGCUUUU | |
| HBG 1/2- | Target | — | CCAUGGGUGGAGUUUAGCCA | SEQ ID |
| first/left gRNA- | Sequence | | | NO: 17 |
| (Pair #8) | | | | |
| HBG 1/2- | gRNA | S. pyogenes | CCAUGGGUGGAGUUUAGCCAGUU | SEQ ID |
| first/left gRNA- | Sequence | | UUAGAGCUAGAAAUAGCAAGUUA | NO: 18 |
| (Pair #8) | | | AAAUAAGGCUAGUCCGUUAUCAA | |
| | | | CUUGAAAAAGUGGCACCGAGUCG | |
| | | | GUGCUUUU | |
| HBG 1/2- | gRNA | S. aureus | AAGGCUGGCCAACCCAUGGGGUU | SEQ ID |
| first/left gRNA- | Sequence | | UUAGUACUCUGGAAACAGAAUCU | NO: 19 |
| (Pair #6) | | | ACUAAAACAAGGCAAAAUGCCGU | |
| | | | GUUUAUCUCGUCAACUUGUUGGC | |
| | | | GAGAUUUU | |

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory, it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)), as shown below:

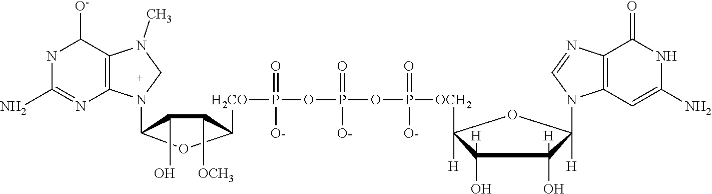

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g., a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

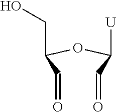

wherein “U” can be an unmodified or modified uridine.

The 3′ terminal U ribose can be modified with a 2′3′ cyclic phosphate as shown below:

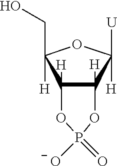

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)n-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In some embodiments, the gRNA comprises one or more chemical modification of a ribonucleotide, a ribonucleotide base, or a phosphodiester bond. In some embodiments, the one or more chemical modification comprises at least one chemically modified phosphodiester bond. In some embodiments, the at least one chemically modified phosphodiester bond is a phosphorothioate bond.

In some embodiments, the gRNA comprises three phosphorothioate bonds at the 5-prime terminus of the gRNA. In some embodiments, the gRNA comprises two phosphorothioate bonds at the 3-primer terminus of the gRNA. In some embodiments, the gRNA comprises a 2′ O-Me chemical modification at the 3′-terminus of the gRNA.

Exemplary gRNA Targeting Sequences

In some embodiments, a gRNA comprises a targeting sequence comprising a nucleotide sequence at least 95%, 96%, 97%, 98%, or 99% (or any percentage in between) identical to SEQ ID NO: 1. In some embodiments, a gRNA comprises a targeting sequence comprising a nucleotide sequence of SEQ ID NO: 1.

In some embodiments, a gRNA comprises a targeting sequence comprising a nucleotide sequence at least 95%, 96%, 97%, 98%, or 99% (or any percentage in between) identical to SEQ ID NO: 3. In some embodiments, a gRNA comprises a targeting sequence comprising a nucleotide sequence of SEQ ID NO: 3.

In some embodiments, a gRNA comprises a targeting sequence comprising a nucleotide sequence at least 95%, 96%, 97%, 98%, or 99% (or any percentage in between) identical to SEQ ID NO: 5. In some embodiments, a gRNA comprises a targeting sequence comprising a nucleotide sequence of SEQ ID NO: 5.

In some embodiments, a gRNA comprises a targeting sequence comprising a nucleotide sequence at least 95%, 96%, 97%, 98%, or 99% (or any percentage in between) identical to SEQ ID NO: 7. In some embodiments, a gRNA comprises a targeting sequence comprising a nucleotide sequence of SEQ ID NO: 7.

In some embodiments, a gRNA comprises a targeting sequence comprising a nucleotide sequence at least 95%, 96%, 97%, 98%, or 99% (or any percentage in between) identical to SEQ ID NO: 9. In some embodiments, a gRNA comprises a targeting sequence comprising a nucleotide sequence of SEQ ID NO: 9.

In some embodiments, a gRNA comprises a targeting sequence comprising a nucleotide sequence at least 95%, 96%, 97%, 98%, or 99% or (or any percentage in between) identical to SEQ ID NO: 11. In some embodiments, a gRNA comprises a targeting sequence comprising a nucleotide sequence of SEQ ID NO: 11.

In some embodiments, a gRNA comprises a targeting sequence comprising a nucleotide sequence at least 95%, 96%, 97%, 98%, or 99% (or any percentage in between) identical to SEQ ID NO: 13. In some embodiments, a gRNA comprises a targeting sequence comprising a nucleotide sequence of SEQ ID NO: 13.

In some embodiments, a gRNA comprises a targeting sequence comprising a nucleotide sequence at least 95%, 96%, 97%, 98%, or 99% (or any percentage in between) identical to SEQ ID NO: 15. In some embodiments, a gRNA comprises a targeting sequence comprising a nucleotide sequence of SEQ ID NO: 15.

In some embodiments, a gRNA comprises a targeting sequence comprising a nucleotide sequence at least 95%, 96%, 97%, 98%, or 99% (or any percentage in between) identical to SEQ ID NO: 17. In some embodiments, a gRNA comprises a targeting sequence comprising a nucleotide sequence of SEQ ID NO: 17.

Exemplary gRNA Sequences

In some embodiments, a gRNA comprises a nucleotide sequence at least 95%, 96%, 97%, 98%, or 99% (or any percentage in between) identical to SEQ ID NO: 2. In some embodiments, a gRNA comprises a nucleotide sequence of SEQ ID NO: 2.

In some embodiments, a gRNA comprises a nucleotide sequence at least 95%, 96%, 97%, 98%, or 99% (or any percentage in between) identical to SEQ ID NO: 4. In some embodiments, a gRNA comprises a nucleotide sequence of SEQ ID NO: 4.

In some embodiments, a gRNA comprises a nucleotide sequence at least 95%, 96%, 97%, 98%, or 99% (or any percentage in between) identical to SEQ ID NO: 6. In some embodiments, a gRNA comprises a nucleotide sequence of SEQ ID NO: 6.

In some embodiments, a gRNA comprises a nucleotide sequence at least 95%, 96%, 97%, 98%, or 99% (or any percentage in between) identical to SEQ ID NO: 8. In some embodiments, a gRNA comprises a nucleotide sequence of SEQ ID NO: 8.

In some embodiments, a gRNA comprises a nucleotide sequence at least 95%, 96%, 97%, 98%, or 99% (or any percentage in between) identical to SEQ ID NO: 10. In some embodiments, a gRNA comprises a nucleotide sequence of SEQ ID NO: 10.

In some embodiments, a gRNA comprises a nucleotide sequence at least 95%, 96%, 97%, 98%, or 99% (or any percentage in between) identical to SEQ ID NO: 12. In some embodiments, a gRNA comprises a nucleotide sequence of SEQ ID NO: 12.

In some embodiments, a gRNA comprises a nucleotide sequence at least 95%, 96%, 97%, 98%, or 99% (or any percentage in between) identical to SEQ ID NO: 14. In some embodiments, a gRNA comprises a nucleotide sequence of SEQ ID NO: 14.

In some embodiments, a gRNA comprises a nucleotide sequence at least 95%, 96%, 97%, 98%, or 99% (or any percentage in between) identical to SEQ ID NO: 16. In some embodiments, a gRNA comprises a nucleotide sequence of SEQ ID NO: 16.

In some embodiments, a gRNA comprises a nucleotide sequence at least 95%, 96%, 97%, 98%, or 99% (or any percentage in between) identical to SEQ ID NO: 19. In some embodiments, a gRNA comprises a nucleotide sequence of SEQ ID NO: 19.

Exemplary gRNA Compositions

In certain compositions of the disclosure, the composition comprises a first gRNA (also referred to as a “left gRNA”) and a second gRNA (also referred to as a “right gRNA”). The first gRNA may comprise a first targeting sequence. The second gRNA may comprise a second targeting sequence.

In some embodiments, the composition comprises a first gRNA comprising a first targeting sequence comprising a nucleotide sequence of SEQ ID NO: 1 and a second gRNA comprising a second targeting sequence comprising a nucleotide sequence of SEQ ID NO: 3. In some embodiments, the composition comprises first gRNA comprising a nucleotide sequence of SEQ ID NO: 2 and a second gRNA comprising a nucleotide sequence of SEQ ID NO: 4.

In some embodiments, the composition comprises a first gRNA comprising a first targeting sequence comprising a nucleotide sequence of SEQ ID NO: 5 and a second gRNA comprising a second targeting sequence comprising a nucleotide sequence of SEQ ID NO: 3. In some embodiments, the composition comprises first gRNA comprising a nucleotide sequence of SEQ ID NO: 6 and a second gRNA comprising a nucleotide sequence of SEQ ID NO: 4.

In some embodiments, the composition comprises a first gRNA comprising a first targeting sequence comprising a nucleotide sequence of SEQ ID NO: 7 and a second gRNA comprising a second targeting sequence comprising a nucleotide sequence of SEQ ID NO: 3. In some embodiments, the composition comprises first gRNA comprising a nucleotide sequence of SEQ ID NO: 8 and a second gRNA comprising a nucleotide sequence of SEQ ID NO: 4.

In some embodiments, the composition comprises a first gRNA comprising a first targeting sequence comprising a nucleotide sequence of SEQ ID NO: 9 and a second gRNA comprising a second targeting sequence comprising a nucleotide sequence of SEQ ID NO: 11. In some embodiments, the composition comprises first gRNA comprising a nucleotide sequence of SEQ ID NO: 10 and a second gRNA comprising a nucleotide sequence of SEQ ID NO: 12.

In some embodiments, the composition comprises a first gRNA comprising a first targeting sequence comprising a nucleotide sequence of SEQ ID NO: 13 and a second gRNA comprising a second targeting sequence comprising a nucleotide sequence of SEQ ID NO: 11. In some embodiments, the composition comprises first gRNA comprising a nucleotide sequence of SEQ ID NO: 14 and a second gRNA comprising a nucleotide sequence of SEQ ID NO: 12.

In some embodiments, the composition comprises a first gRNA comprising a first targeting sequence comprising a nucleotide sequence of SEQ ID NO: 7 and a second gRNA comprises a second targeting sequence comprising a nucleotide sequence of SEQ ID NO: 11. In some embodiments, the composition comprises first gRNA comprising a nucleotide sequence of SEQ ID NO: 19 and a second gRNA comprising a nucleotide sequence of SEQ ID NO: 12.

In some embodiments, the composition comprises a first gRNA comprises a first targeting sequence comprising a nucleotide sequence of SEQ ID NO: 15 and a second gRNA comprising comprises a second targeting sequence comprising a nucleotide sequence of SEQ ID NO: 3. In some embodiments, the composition comprises first gRNA comprising a nucleotide sequence of SEQ ID NO: 16 and a second gRNA comprising a nucleotide sequence of SEQ ID NO: 4.

In some embodiments, the composition comprises a first gRNA comprising a first targeting sequence comprising a nucleotide sequence of SEQ ID NO: 17 and a second gRNA comprising a second targeting sequence comprising a nucleotide sequence of SEQ ID NO: 3. In some embodiments, the composition comprises first gRNA comprising a nucleotide sequence of SEQ ID NO: 18 and a second gRNA comprising a nucleotide sequence of SEQ ID NO: 4.

Exemplary Cas-CLOVER and gRNA Compositions

Gene editing compositions, including Cas-CLOVER, and methods of using these compositions for gene editing are described in detail in PCT Application Numbers PCT/US2016/037922, PCT/US2018/066941, PCT/US2017/054799, U.S. Patent Publication Nos. 2017/0107541, 2017/0114149, 2018/0187185 and U.S. Pat. No. 10,415,024, each of which are incorporated herein by reference in its entirety for examples of gene editing compositions that may be used in the methods disclosed herein. Exemplary gene editing compositions including Cas-CLOVER and methods of using these compositions for gene editing are described herein.

In certain compositions of the disclosure, the composition comprises a first gRNA (also referred to as a “left gRNA”), a first fusion protein or a first polynucleotide encoding the first fusion protein (e.g. Cas-Clover), a second gRNA (also referred to as a “right gRNA”) and a second fusion protein or a second polynucleotide encoding a second fusion protein (e.g., Cas-Clover). The first gRNA comprises a first targeting sequence. The second gRNA comprises a second targeting sequence.

In some embodiments, the first gRNA and the first fusion protein is complexed at the 5′ terminus of the target DNA to be modified. In some embodiments, the second gRNA and the second fusion protein is complexed at the 3′ terminus of the target DNA to be modified. A schematic diagram of the composition complexed with a target DNA is shown in FIG. 7.

In some embodiments, the first fusion protein, the second fusion protein, or both the first and the second fusion protein comprises a dCas9 derived from a S. pyogenes Cas9 polypeptide. In some embodiments, the first fusion protein, the second fusion protein, or both the first and the second fusion protein comprises a dCas9 derived from a S. aureus Cas9 polypeptide. Exemplary compositions of the disclosure are shown in Table 2.

TABLE 2
Exemplary Compositions of the Disclosure

| | | | | | Second | |
|---|---|---|---|---|---|---|
| | First | First (left) | | Second | (right) | Second |
| | (left) | gRNA | First (left) | (right) | gRNA | (right) |
| Composition | gRNA | Full | Cas- | gRNA | Full | Cas- |
| Name | Template | Sequence | Clover | Template | sequence | Clover |
| Pair #1 | SEQ ID | SEQ ID | SEQ ID | SEQ ID | SEQ ID | SEQ ID |
| | NO: 1 | NO: 2 | NO: 39 | NO: 3 | NO: 4 | NO: 39 |
| Pair #2 | SEQ ID | SEQ ID | SEQ ID | SEQ ID | SEQ ID | SEQ ID |
| | NO: 5 | NO: 6 | NO: 39 | NO: 3 | NO: 4 | NO: 39 |
| Pair #3 | SEQ ID | SEQ ID | SEQ ID | SEQ ID | SEQ ID | SEQ ID |
| | NO: 7 | NO: 8 | NO: 41 | NO: 3 | NO: 4 | NO: 41 |
| Pair #4 | SEQ ID | SEQ ID | SEQ ID | SEQ ID | SEQ ID | SEQ ID |
| | NO: 9 | NO: 10 | NO: 39 | NO: 11 | NO: 12 | NO: 42 |
| Pair #5 | SEQ ID | SEQ ID | SEQ ID | SEQ ID | SEQ ID | SEQ ID |
| | NO: 13 | NO: 14 | NO: 42 | NO: 11 | NO: 12 | NO: 42 |
| Pair #6 | SEQ ID | SEQ ID | SEQ ID | SEQ ID | SEQ ID | SEQ ID |
| | NO: 7 | NO: 19 | NO: 42 | NO: 11 | NO: 12 | NO: 42 |
| Pair #7 | SEQ ID | SEQ ID | SEQ ID | SEQ ID | SEQ ID | SEQ ID |
| | NO: 15 | NO: 16 | NO: 41 | NO: 3 | NO: 4 | NO: 41 |
| Pair #8 | SEQ ID | SEQ ID | SEQ ID | SEQ ID | SEQ ID | SEQ ID |
| | NO: 17 | NO: 18 | NO: 41 | NO: 3 | NO: 4 | NO: 41 |

In some embodiments, the composition comprises first gRNA comprising a nucleotide sequence of SEQ ID NO: 2, a first fusion protein comprising the polypeptide sequence of SEQ ID NO: 39, a second gRNA comprising a nucleotide sequence of SEQ ID NO: 4 and a second fusion protein comprising the polypeptide sequence of SEQ ID NO: 39.

In some embodiments, the composition comprises first gRNA comprising a nucleotide sequence of SEQ ID NO: 6, a first fusion protein comprising the polypeptide sequence of SEQ ID NO: 39, a second gRNA comprising a nucleotide sequence of SEQ ID NO: 4 and a second fusion protein comprising the polypeptide sequence of SEQ ID NO: 39.

In some embodiments, the composition comprises first gRNA comprising a nucleotide sequence of SEQ ID NO: 8, a first fusion protein comprising the polypeptide sequence of SEQ ID NO: 41, a second gRNA comprising a nucleotide sequence of SEQ ID NO: 4 and a second fusion protein comprising the polypeptide sequence of SEQ ID NO: 41.

In some embodiments, the composition comprises first gRNA comprising a nucleotide sequence of SEQ ID NO: 10, a first fusion protein comprising the polypeptide sequence of SEQ ID NO: 39, a second gRNA comprising a nucleotide sequence of SEQ ID NO: 12 and a second fusion protein comprising the polypeptide sequence of SEQ ID NO: 42.

In some embodiments, the composition comprises first gRNA comprising a nucleotide sequence of SEQ ID NO: 14, a first fusion protein comprising the polypeptide sequence of SEQ ID NO: 42, a second gRNA comprising a nucleotide sequence of SEQ ID NO: 12 and a second fusion protein comprising the polypeptide sequence of SEQ ID NO: 42.

In some embodiments, the composition comprises first gRNA comprising a nucleotide sequence of SEQ ID NO: 19, a first fusion protein comprising the polypeptide sequence of SEQ ID NO: 42, a second gRNA comprising a nucleotide sequence of SEQ ID NO: 12 and a second fusion protein comprising the polypeptide sequence of SEQ ID NO: 42.

In some embodiments, the composition comprises first gRNA comprising a nucleotide sequence of SEQ ID NO: 16, a first fusion protein comprising the polypeptide sequence of SEQ ID NO: 41, a second gRNA comprising a nucleotide sequence of SEQ ID NO: 4 and a second fusion protein comprising the polypeptide sequence of SEQ ID NO: 41.

In some embodiments, the composition comprises first gRNA comprising a nucleotide sequence of SEQ ID NO: 18, a first fusion protein comprising the polypeptide sequence of SEQ ID NO: 41, a second gRNA comprising a nucleotide sequence of SEQ ID NO: 4 and a second fusion protein comprising the polypeptide sequence of SEQ ID NO: 41.

Delivery of gRNAs and Genetic Editing Compositions

Gene editing tools can also be delivered to cells using one or more poly(histidine)-based micelles. Poly(histidine) (e.g., poly(L-histidine)), is a pH-sensitive polymer due to the imidazole ring providing an electron lone pair on the unsaturated nitrogen. That is, poly(histidine) has amphoteric properties through protonation-deprotonation. In particular, at certain pHs, poly(histidine)-containing triblock copolymers may assemble into a micelle with positively charged poly(histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA is enabled by delivery of a nuclease using the poly(histidine)-based micelles. Without wishing to be bound by a particular theory, it is believed that believed that in the micelles that are formed by the various triblock copolymers, the hydrophobic blocks aggregate to form a core, leaving the hydrophilic blocks and poly(histidine) blocks on the ends to form one or more surrounding layer.

In an aspect, the disclosure provides triblock copolymers made of a hydrophilic block, a hydrophobic block, and a charged block. In some aspects, the hydrophilic block may be poly(ethylene oxide) (PEO), and the charged block may be poly(L-histidine). An example tri-block copolymer that can be used is a PEO-b-PLA-b-PHIS, with variable numbers of repeating units in each block varying by design.

Diblock copolymers that can be used as intermediates for making triblock copolymers can have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ε-caprolactone) (PCL), and poly (trimethylene carbonate) (PTMC). Polymeric micelles comprised of 100% PEGylated surfaces possess improved in vitro chemical stability, augmented in vivo bioavailablity, and prolonged blood circulatory half-lives.

Polymeric vesicles, polymersomes and poly(Histidine)-based micelles, including those that comprise triblock copolymers, and methods of making the same, are described in further detail in U.S. Pat. Nos. 7,217,427; 7,868,512; 6,835,394; 8,808,748; 10,456,452; U.S. Publication Nos. 2014/0363496; 2017/0000743; and 2019/0255191; and PCT Publication No. WO 2019/126589, each of which are incorporated herein by reference in its entirety for examples of polymers that may be used to deliver the compositions disclosed herein.

Gene editing compositions (e.g. mutant Cas-Clover) can also be delivered to cells using one or more lipid nanoparticle compositions and methods of making the same, as described in PCT Publication Nos. WO 2022/182792 and WO 2023/141576, each of which is incorporated herein by reference in its entirety for examples of lipid nanoparticles that may be used to deliver the gene editing compositions disclosed herein.

In some aspects, the composition is encapsulated in at least one lipid nanoparticle comprising: about 40.75% of a terpene lipidoid compound by moles, about 51.75% of cholesterol by moles, about 5% of DOPC by moles, and about 2.5% of DMG-PEG2000 by moles, wherein a polynucleotide encoding the mutant Cas-Clover is a RNA molecule, and wherein the ratio of lipid to RNA molecule in the at least one nanoparticle is about 120:1 (w/w).

In some aspects, the terpene lipidoid compound is HMA-404:

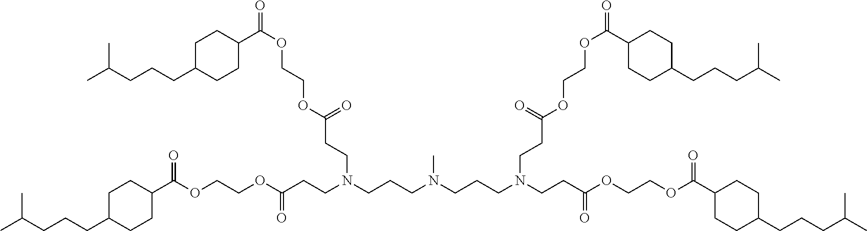 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Accordingly, in some aspects, the gene editing composition is encapsulated in at least one lipid nanoparticle comprising: about 40.75% of HMA-404 by moles, about 51.75% of cholesterol by moles, about 5% of DOPC by moles, and about 2.5% of DMG-PEG2000 by moles, wherein a polynucleotide encoding the mutant Cas-Clover is a RNA molecule, and wherein the ratio of lipid to RNA molecule in the at least one nanoparticle is about 120:1 (w/w).

In some aspects, the composition is encapsulated in at least one lipid nanoparticle comprising: about 54% of SS-OP by moles, about 35% of cholesterol by moles, about 5% of DOPC by moles, about 5% of DSPC by moles, and about 1% of DMG-PEG2000 by moles. The ratio of lipid to nucleic acid in the nanoparticles was about 100:1 (weight/weight) and the total lipid of 25 mM.

### Cells and Modified Cells of the Disclosure

Cells and modified cells of the disclosure can be mammalian cells. The cells and modified cells are human cells. In some embodiments, the cells can comprise hematopoietic progenitor cells (HPCs). In some embodiments, the cells can comprise Hematopoietic stem cells (HSCs). In some embodiments, the cells are hematopoietic stem and precursor cells (HSPCs). In some embodiments, the HSPCs are capable of differentiating into erythroid progenitor cells. In certain embodiments, at least a portion of the plurality of cells may be within an erythroid lineage. In some embodiments, the HSPC is capable of differentiating into an erythroid progenitor cell.

Cells that have been altered ex vivo according to this disclosure can be manipulated (e.g., expanded, passaged, frozen, differentiated, de-differentiated, transduced with a transgene, etc.) prior to their delivery to a subject. The cells are, variously, delivered to a subject from which they are obtained (in an “autologous” transplant), or to a recipient who is immunologically distinct from a donor of the cells (in an “allogeneic” transplant).

In some embodiments, an autologous transplant includes the steps of obtaining, from the subject, a plurality of cells, either circulating in peripheral blood, or within the marrow or other tissue (e.g., spleen, skin, etc.), and manipulating those cells to enrich for cells in the erythroid lineage (e.g., by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system targeting BCL11A gene or HBG1 and/or HBG2 promoter target sequence. The genome editing system can be implemented or delivered to the cells in any suitable format, including as a ribonucleoprotein complex, as separated protein and nucleic acid components, and/or as nucleic acids encoding the components of the genome editing system.

The cells, following delivery of the genome editing system, are optionally manipulated e.g., to enrich for HSCs and/or cells in the erythroid lineage and/or for edited cells, to expand them, freeze/thaw, or otherwise prepare the cells for return to the subject. The edited cells are then returned to the subject, for instance in the circulatory system by means of intravenous delivery or delivery or into a solid tissue such as bone marrow.

### Modified Cells of the Disclosure

The disclosure provides a method of modifying a population of cells comprising contacting the population of cells with the compositions of the disclosure (e.g., first Cas-Clover fusion protein and first gRNA, and second Cas-Clover fusion protein and second gRNA compositions), wherein the first gRNA forms a complex with the first targeting sequence and the first fusion protein, and the second gRNA forms a complex with the second targeting sequence and the second fusion protein, thereby generating an insertion or deletion (indel) between the first targeting sequence and the second targeting sequence and producing a modified population of cells. In some embodiments, the indel is generated at the BCL11A gene, HMG1 promoter region, HMG2 promoter region, or a combination thereof. In some embodiments, the indel causes inactivation of the BCL11A gene.

In some embodiments, the disclosure relates to compositions including a plurality of cells generated by the method disclosed above, in which at least 20%, 30%, 40%, 50%, 60%, 70%, 80% or 90% of the cells include an indel between the first targeting sequence and the second targeting sequence. In some embodiments, the disclosure relates to compositions including a plurality of cells generated by the method disclosed above, in which at least 20%, 30%, 40%, 50%, 60%, 70%, 80% or 90% of the cells include an indel at the BCL11A gene. In some embodiments, the disclosure relates to compositions including a plurality of cells generated by the method disclosed above, in which at least 20%, 30%, 40%, 50%, 60%, 70%, 80% or 90% of the cells include an indel at the HMG1 promoter region. In some embodiments, the disclosure relates to compositions including a plurality of cells generated by the method disclosed above, in which at least 20%, 30%, 40%, 50%, 60%, 70%, 80% or 90% of the cells include an indel at the HMG2 promoter region.

In certain embodiments, the plurality of cells may be characterized by an increased level of fetal hemoglobin (HbF) expression relative to an unmodified plurality of cells. In certain embodiments, the level of fetal hemoglobin may be increased by at least 20%, 30%, 40%, 50%, 60%, 70%, 80% or 90%. In certain embodiments, the level of fetal hemoglobin may be increased by about 1 fold to about 10 fold. In certain embodiments, the level of fetal hemoglobin may be increased by about 4 fold to about 9 fold. In certain embodiments, the level of fetal hemoglobin may be increased by about 1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10 fold. In certain embodiments, the level of fetal hemoglobin may be increased by about 1-fold, about 2-fold, about 3-fold, about 4-fold, about 5-fold, about 6-fold, about 7-fold, about 8-fold, about 9-fold, or about 10-fold.

In certain embodiments, the plurality of cells may be characterized by an increased level of gamma globulin expression relative to an unmodified plurality of cells. In certain embodiments, the level of gamma globulin may be increased by at least 20%, 30%, 40%, 50%, 60%, 70%, 80% or 90%. In certain embodiments, the level of gamma globulin may be increased by about 1-fold to about 10-fold. In certain embodiments, the level of gamma globulin may be increased by about 4-fold to about 9-fold. In certain embodiments, the level of gamma globulin may be increased by about 1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold. In certain embodiments, the level of gamma globulin may be increased by about 1-fold, about 2-fold, about 3-fold, about 4-fold, about 5-fold, about 6-fold, about 7-fold, about 8-fold, about 9-fold, or about 10-fold.

Cells and modified immune cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

In certain aspects, the cells of the present disclosure are modified to recombinantly express dihydrofolate reductase (DHFR), which advantageously renders cells resistant to methotrexate (MTX). The MTX resistant cells may be used in methods of treating a subject in need thereof in combination with subsequent MTX administration to eliminate activated T-cells and NK cells targeting the modified cells or therapeutic cells, thereby increasing the in vivo persistence and efficacy of the modified cells.

### Formulations, Dosages and Modes of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically, the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Methods of Using the Compositions of the Disclosure

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for modulating or treating at least one disease or disorder in a cell, tissue, organ, animal or subject. Preferably, the malignant disease is a beta-hemoglobinopathy. Non-limiting examples of a beta-hemoglobinopathies include sickle cell disease and beta-thalassemia.

The compositions of the disclosure may be used to treat a disease or disorder by use of a therapeutic transgene encoding for an exogenous nucleic acid sequence or exogenous amino acid sequence. For certain diseases or disorders the therapeutic transgene can include [Disease](therapeutic transgene): [Beta-Thalassemia](HBB T87Q, BCL11A shRNA, IGF2BP1), [Sickle Cell Disease](HBB T87Q, BCL11A shRNA, IGF2BP1.

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically, the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

The term “binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof, (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 3, Table 4 and Table 5 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 3.

TABLE 3
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 4.

TABLE 4
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 5.

TABLE 5
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34+ hematopoietic cells capable of giving rise to both mature myeloid and lymphoid cell types including T cells, NK cells and B cells. Hematopoietic cells also include various subsets of primitive hematopoietic cells that give rise to primitive erythrocytes, megakarocytes and macrophages.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, tissue, biopsy. The term also includes a cell that is removed from at least one, some or all components as the cell is found in non-naturally occurring environments, for example, culture, cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cells, substantially pure cells and cells cultured in a medium that is non-naturally occurring. Isolated cells may be obtained from separating the desired cells, or populations thereof, from other substances or cells in the environment, or from removing one or more other cell populations or subpopulations from the environment. As used herein, the term “purify” or the like refers to increase purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” in intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof, a variant polypeptide (i.e. a mutant of the native polypeptide having less than 100% sequence identity with the native polypeptide) or fragment thereof, an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “engager” refers to a molecule, e.g. a fusion polypeptide, which is capable of forming a link between an immune cell, e.g. a T cell, a NK cell, a NKT cell, a B cell, a macrophage, a neutrophil, and a tumor cell; and activating the immune cell. Examples of engagers include, but are not limited to, bi-specific T cell engagers (BiTEs), bi-specific killer cell engagers (BiKEs), tri-specific killer cell engagers, or multi-specific killer cell engagers, or universal engagers compatible with multiple immune cell types.

As used herein, the term “specific” or “specificity” can be used to refer to the ability of a molecule, e.g., a receptor or an engager, to selectively bind to a target molecule, in constrast to non-specific or non-selective binding.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level—successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level—successful removal, adding, or altering a cell function/characteristics via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

### EXAMPLES

The Examples in this section are provided for illustration and are not intended to limit the invention.

### Example 1: Methods of Genetically Engineering Hematopoietic Stem and Progenitor Cells

Exemplary Method for Isolation of Human Hematopoietic Stem and Progenitor Cells (HSPCs) from Peripheral Blood

Human CD34⁺ HSPCs were isolated from mobilized peripheral blood of healthy donors using a Miltenyi Biotec automated CliniMACS Prodigy Instrument with CD34 GMP MicroBeads (Miltenyi Biotec, #170-076-711) and the TS 510 program in accordance with the manufacturer's instructions. Purity of isolated cells was assessed via flow cytometry using CD34-AF647 (BioLegend, Clone 561, #343618) and CD45-PE (BioLegend, Clone HI30, #304008) with DAPI (BioLegend, #422801) as the viability dye. On the same day as isolation, cells were frozen in CryoStor CS10 medium (STEMCELL Technologies, #07930).

Exemplary Method for Introducing Nucleic Acids into HSPCs Using Electroporation

Frozen CD34⁺ HSPCs isolated using the methods described above were thawed and cultured in StemSpan SFEM II (STEMCELL Technologies, #09655) medium supplemented with 100 ng/mL recombinant human Stem Cell Factor, 100 ng/mL recombinant human Flt3/Flk-2 Ligand, and 100 ng/mL recombinant human thrombopoietin (STEMCELL Technologies Cat. Nos. 78062, 78009, and 78210.1, respectively) at 1×10⁶ cells/mL in a humidified incubator with 5% CO₂ atmosphere at 37° C. After 24 hours in culture, HSPCs were subject to electroporation using P3 Primary Cell 4D-Nucleofector X Kit (Lonza, #V4XP-3032) with program EO-100 in accordance with the manufacturer's instructions. Electroporated cells were resuspended in culture medium at 7.5×10⁵ cells/mL. One day after electroporation, HSPCs underwent a complete medium change and were cultured for an additional 3 days for further downstream analysis and assays.

### Exemplary Method for Erythroid Differentiation of HSPCs

Four days after electroporation, HSPCs were switched to an expansion medium consisting of StemSpan SFEM II medium supplemented with 10 ng/mL recombinant human Stem Cell Factor, 2 U/mL recombinant human erythropoietin (STEMCELL Technologies, #78007.1), 1 ng/mL human IL-3 (R&D Systems, #203-IL-010), 1 mM dexamethasone (Sigma, #D4902), and 1 mM β-estradiol (BioGems, #5022822) at a density of 2×10⁵ cells/mL. Every three to four days, cells were counted, and expansion medium was added to the culture to maintain cells at 2×10⁵ cells/mL. After 11 days in expansion medium, cells were subject to a complete medium change into a maturation medium consisting of StemSpan SFEM II supplemented with 4 U/mL recombinant human erythropoietin and 0.5 mg/mL holo-transferrin (Sigma, #T0665) at a density of 4 to 5×10⁶ cells/mL. Cells were maintained in maturation medium for an additional 7 days. Every three to four days, cells were counted, and maturation medium added to the culture to maintain cells at 4-6×10⁶ cells/mL.

### Example 2: Methods for Analyzing Genetically Edited Hematopoietic Stem and Progenitor Cells

### Exemplary Method for Detection and Quantification of Insertions or Deletions (Indels) in HBG Gene Edited HSPCs and Erythroid Differentiated Cells

Genomic DNA (gDNA) was extracted from control and HBG gene edited HSPCs using the Quick-DNA Microprep Kit (Zymo, #D3020) in accordance with the manufacturer's instructions. The extracted genomic DNA was subject to PCR amplification of the HBG1/2 genes using the Platinum™ SuperFi II PCR Master Mix (ThermoFisher, #12368050) in accordance with the manufacturer's instructions using a forward primer:

ACACTCTTTCCCTACACGACGCTCTTCCGATCTGCAGTATCCTCTTGGGGG (SEQ ID NO: 24), and a reverse primer:
GACTGGAGTTCAGACGTGTGCTCTTCCGATCTACCTCAGACGTTCCAGAAGC (SEQ ID NO: 25) flanking the HBG gene region of interest.

PCR products were further purified using the Select-a-Size DNA Clean & Concentrator MagBead Kit (Zymo, #D4085) in accordance with the manufacturer's instructions followed by next-generation sequencing (NGS) library preparation using the NEBNext® Ultra™ II Q5@Master Mix (New England BioLabs, #M0544X) with the NEBNext® Multiplex Oligos for Illumina® (96 Index Primers) (New England BioLabs, #E6609S) for sample multiplexing. Libraries were pooled and pair-end sequenced on the Illumina MiSeq with a depth of at least 20,000 reads per sample in accordance with the manufacturer's instructions. Generated FASTQ files were used as input for indel quantification using the CRISPResso2 software with the following options: −min_average_read_quality 30, −min_single_bp_quality 10, −exclude bp_from_left 15, −exclude bp_from_right 15, −ignore_substitutions TRUE, −amplicon_min_alignment_score 60, −quantification_window_size 90.

For separate indel measurement at the individual HBG1 or HBG2 locus, a pre-amplification step was added to the above-described procedures using the Platinum™ SuperFi II PCR Master Mix with the following PCR primers.
HBG1 forward: TCCACAGTACCTGCCAAAGA (SEQ ID NO: 26);
HBG1 reverse: GCCTACCTTCCCAGGGTTTC (SEQ ID NO: 27);
HBG2 forward: GGCCTAAAACCACAGAGAGTAT (SEQ ID NO: 28); and
HBG2 reverse: CCCCACAGGCTTGTGATAGT (SEQ ID NO: 29). Indel measurement at the individual HBG1 or HBG 2 locus was determined using the methods described above.

### Exemplary Method to Analyze and Quantify HSPC and Erythroid Progenitor Populations Using Flow Cytometry

Prior to erythroid differentiation, HSPCs were subject to flow cytometry to assess their stemness marker expression, including CD34-BV785 (BioLegend, Clone 561, #343626), CD90-APC (BioLegend, Clone 5E10, #328114), CD38-PE (BioLegend, Clone HB-7, #356604) and CD133-PE (BD, Clone 293C3, #567917), along with a viability dye Fixable Viability Stain 450 (BD, #562247). Cells analyzed during erythroid differentiation were harvested and cell surface stained with the erythroid lineage markers CD235a-APC (BioLegend, Clone HI264, #349114) and CD71-PerCP/Cy5.5 (BioLegend, Clone CY1G4, #334114), along with the viability dye Fixable Viability Stain 450. Subsequently, differentiated cells were fixed and permeabilized using the Transcription Factor Buffer Set (BD, #562725) followed by intracellular staining of fetal hemoglobin using HbF-PE (BD, Clone 2D12, #560041). Stained cells were measured on BD FACS Celesta or Agilent NovoCyte Quanteon in accordance with the manufacturer's instructions.

Exemplary Method for Measuring Percent Indels Gamma Globin Gene Expression in Cells Differentiated from Unedited and HBG Edited HSPCs Using RT-qPCR.

HSPC differentiated cells were harvested and mRNA extracted using the RNeasy Mini Kit (QIAGEN, #74106) with on-column DNA digestion in accordance with the manufacturer's instructions (RNase-Free DNase Set, QIAGEN, #79254). mRNA was reverse transcribed using the iScript™ Reverse Transcription Supermix (Bio-Rad, #1708841). The resulting cDNA was used for qPCR using the SsoAdvanced Universal Probes Supermix (Bio-Rad, #1725281) on the Bio-Rad CFX Opus 384 Real-Time PCR Instrument in accordance with the manufacturer's instructions. TaqMan probe-based assays were ordered from ThermoFisher to detect gene expression of HBG1/2 (Hs00361131_g1), HBB (Hs00747223_g1), HBA1 (Hs07292163_s1) and RPP30 (Hs01124518_ml).

Exemplary Method for Analyzing and Quantitating Cells Differentiated from Unedited and HBG Edited HSPC Cells Using a Colony-forming Unit (CFU) Assay

At 4 days post-electroporation, control and edited HSPCs were frozen in CryoStor CS10 medium. Frozen HSPCs were utilized in a colony-forming unit (CFU) assay in accordance with manufacturer's instructions. Upon thawing, cells were added to an aliquot of MethoCult™ GF+H4435 (H4435 Enriched) (STEMCELL Technologies, #04445). To maximize the number of well-separated colonies to form, cells were plated at 3 densities (250, 500 and 1500 cells/well) with each density having 3 replicate wells on a 6-well STEMvision™ SmartDish™ (STEMCELL Technologies, #27371). Cells were placed in a humidified incubator with 5% CO₂ atmosphere at 37° C. for 12-14 days. Subsequently, the total number of erythroid (BFU-E), myeloid (CFU-GM) and multi-potential progenitor (CFU-GEMM) colonies were evaluated by trained personnel and enumerated based on morphology. For each sample, well-separated BFU-E and CFU-GM colonies were harvested from the cultures and genomic DNA from the harvested colonies was extracted using the QuickExtract DNA Extraction Solution (Biosearch Technologies, #QE0905T). Remaining colonies from each of the harvested cultures also were harvested in bulk and genomic DNA extracted using the Quick-DNA Microprep Kit. gDNA from both individual colonies and bulk culture was subject to indel detection and quantification.

### Example 3: Compositions Comprising Cas-CLOVER and Exemplary HBG gRNA Pairs Result in Dose-dependent Editing of HBG loci in HSPCs

Exemplary compositions comprising Cas-CLOVER and gRNA pairs were designed to edit the BCL11A binding sites present upstream of the HBG1 and HBG2 genes in HSPCs. HSPCs were isolated and frozen according to the methods described above. HSPCs were then thawed and cultured for 24 hours according to the methods described above. After 24 hours in culture, HSPCs were nucleofected with mRNA encoding Cas-CLOVER and gRNA pairs according to Table 6.

TABLE 6
Cas-CLOVER and gRNA pairs targeting HBG1 or HBG2

| | Cas- | | gRNA pair |
|---|---|---|---|
| | CLOVER | | concentrations |
| Cas-CLOVER | (μg/ml) | gRNA pairs | (μg/ml) |
| SEQ ID NO: 39 | 100 | Pair #1 | 40, 200, 400 |
| | | SEQ ID NO: 2 and SEQ ID NO: 4) | |
| SEQ ID NO: 39 | 100 | Pair #2 | 40, 200, 400 |
| | | (SEQ ID NO: 6 and SEQ ID NO: 4) | |
| SEQ ID NO: 41 | 100 | Pair #3 | 40, 200, 400 |
| | | (SEQ ID NO: 8 and SEQ ID NO: 4) | |
| SEQ ID NO: 39 | 50 | Pair #4 | 40, 200, 400 |
| SEQ ID NO: 42 | 50 | (SEQ ID NO: 10 and SEQ ID NO: 12) | |

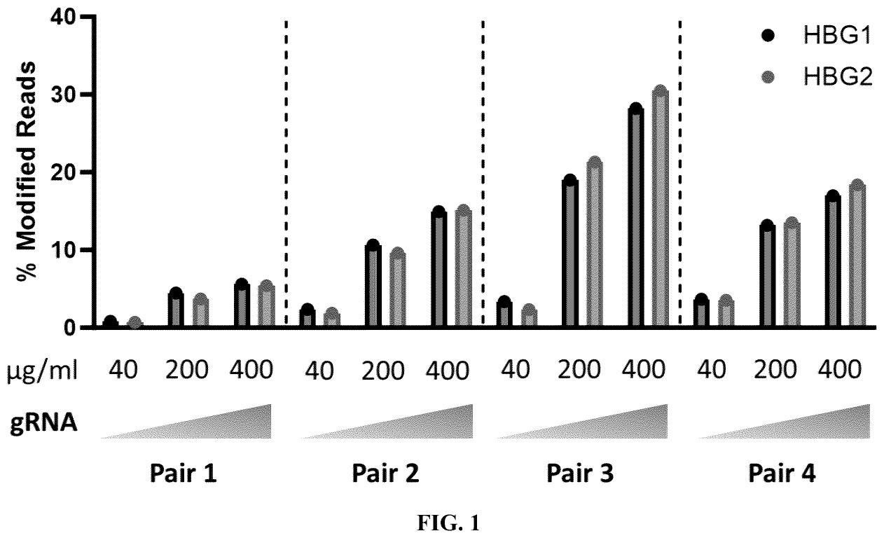

Electroporated cells were resuspended in culture medium at 7.5×10⁵ cells/mL for a period of four days. Four days later, electroporated cells were harvested and genomic DNA (gDNA) was extracted according to the methods described above. The HBG genomic region encompassing the Cas-CLOVER targeted sites was PCR-amplified and subjected to next-generation sequencing (NGS). Small insertions-and-deletions (indels) induced by Cas-CLOVER editing, as measured by percentages of modified reads out of total sequencing reads, were quantified using the CRISPResso2 bioinformatic pipeline described above. All four gRNA pairs generate indels in a dose-dependent manner at both the HBG1 and HBG2 loci in electroporated HSPCs (FIG. 1). The gRNA Pairs #2, #3 and #4 generated significantly higher indels than Pair #1. At a concentration of about 400 μg/ml, Pair #3 resulted in greater than 30% modified reads.

This experimental procedure was repeated using additional gRNA pairs and concentrations. HSPCs were nucleofected with mRNA encoding Cas-CLOVER and gRNA pairs according to Table 7.

TABLE 7
Cas-CLOVER and gRNA pairs targeting HBG1 or HBG2

| | Cas- | | gRNA pair |
|---|---|---|---|
| | CLOVER | | concentrations |
| Cas-CLOVER | (μg/ml) | gRNA pairs | (μg/ml) |
| SEQ ID NO: 39 | 200 | Pair #2 | 200, 400 |
| | | (SEQ ID NO: 6 and SEQ ID NO: 4) | |
| SEQ ID NO: 41 | 200 | Pair #3 | 200, 400 |
| | | (SEQ ID NO: 8 and SEQ ID NO: 4) | |
| SEQ ID NO: 39 | 100 | Pair #4 | 200, 400 |
| SEQ ID NO: 42 | 100 | (SEQ ID NO: 10 and SEQ ID NO: 12) | |
| SEQ ID NO: 42 | 200 | Pair #5 | 200, 400 |
| | | (SEQ ID NO: 14 and SEQ ID NO: 12) | |
| SEQ ID NO: 42 | 200 | Pair #6 | 200, 400 |
| | | (SEQ ID NO: 19 and SEQ ID NO: 12) | |
| SEQ ID NO: 41 | 200 | Pair #7 | 200, 400 |
| | | (SEQ ID NO: 16 and SEQ ID NO: 4) | |
| SEQ ID NO: 41 | 200 | Pair #8 | 200, 400 |
| | | (SEQ ID NO: 18 and SEQ ID NO: 4) | |

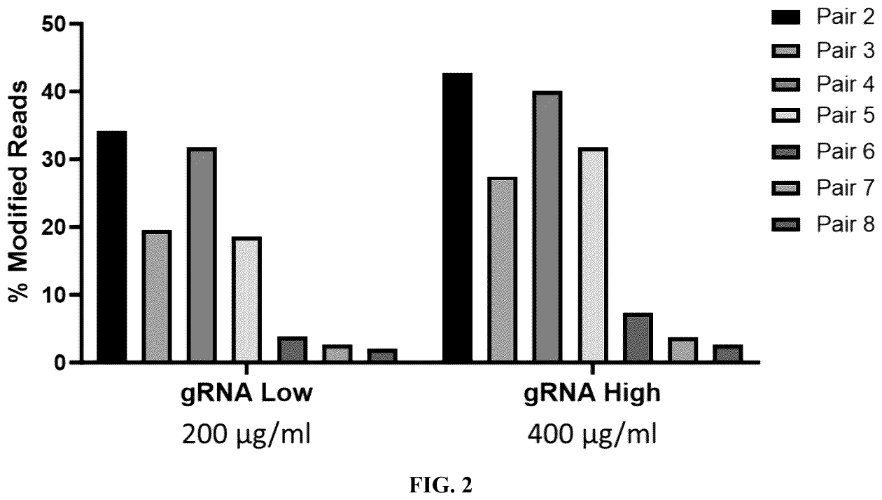

The results of this experiment is shown in FIG. 2. gRNA Pairs #2, #3, #4 and #5 demonstrated appreciable editing of the HBG loci resulting in indel percentages between about 20-35% and a trend of increased editing percentages at higher gRNA concentrations. gRNA Pairs #6, #7 and #8 exhibited reduced levels of HBG editing compared to gRNA Pairs #2, #3, #4 and #5.

This experimental procedure was repeated using certain gRNA pairs and Cas-Clover variants. HSPCs were nucleofected with mRNA encoding Cas-CLOVER (100 ug/mL for each variant) and gRNA pairs (200 ug/mL for each guide) according to Table 7.

TABLE 8
Cas-CLOVER and gRNA pairs targeting HBG1 or HBG2

| Pair | Cas-CLOVER | | |
|---|---|---|---|
| ID | (Left and Right) | | gRNA pairs |
| 4 | SpCC SEQ | SaCC SEQ | SEQ ID NO: 9 |
| | ID NO: 39 | ID NO: 42 | GGCAAGGCTGGCCAACCCAT |
| | | | SEQ ID NO: 11 |
| | | | GCAAACTTGACCAATAGTCT |
| 4.1 | SpCC SEQ | SaCC-XL | SEQ ID NO: 9 |
| | ID NO: 39 | SEQ ID NO: | GGCAAGGCTGGCCAACCCAT |
| | | 43 | SEQ ID NO: 11 |
| | | | GCAAACTTGACCAATAGTCT |
| 9 | SpCC SEQ | SaCC SEQ | SEQ ID NO: 15 |
| | ID NO: 39 | ID NO: 42 | CCCATGGGTGGAGTTTAGCC |
| | | | SEQ ID NO: 11 |
| | | | GCAAACTTGACCAATAGTCT |
| 9.1 | SpCC-XL | SaCC-XL | SEQ ID NO: 15 |
| | SEQ ID NO: | SEQ ID NO: | CCCATGGGTGGAGTTTAGCC |
| | 41 | 43 | SEQ ID NO: 11 |
| | | | GCAAACTTGACCAATAGTCT |
| 9.2 | SpCC | SaCC-XL | SEQ ID NO: 15 |
| | SEQ ID NO: | SEQ ID NO: | CCCATGGGTGGAGTTTAGCC |
| | 39 | 43 | SEQ ID NO: 11 |
| | | | GCAAACTTGACCAATAGTCT |
| 10 | SpCC | SaCC SEQ | SEQ ID NO: 17 |
| | SEQ ID NO: | ID NO: 42 | CCATGGGTGGAGTTTAGCCA |
| | 39 | | SEQ ID NO: 11 |
| | | | GCAAACTTGACCAATAGTCT |
| 10.1 | SpCC-XL | SaCC-XL | SEQ ID NO: 17 |
| | SEQ ID NO: | SEQ ID NO: | CCATGGGTGGAGTTTAGCCA |
| | 41 | 43 | SEQ ID NO: 11 |
| | | | GCAAACTTGACCAATAGTCT |
| 10.2 | SpCC | SaCC-XL | SEQ ID NO: 17 |
| | SEQ ID NO: | SEQ ID NO: | CCATGGGTGGAGTTTAGCCA |
| | 39 | 43 | SEQ ID NO: 11 |
| | | | GCAAACTTGACCAATAGTCT |
| 11 | SaCC SEQ | SpCC | SEQ ID NO: 13 |
| | ID NO: 42 | SEQ ID NO: | AAGGCAAGGCTGGCCAACCC |
| | | 39 | SEQ ID NO: 3 |
| | | | TAGTCTTAGAGTATCCAGTG |
| 11.1 | SaCC-XL | SpCC-XL | SEQ ID NO: 13 |
| | SEQ ID NO: | SEQ ID NO: | AAGGCAAGGCTGGCCAACCC |
| | 43 | 41 | SEQ ID NO: 3 |
| | | | TAGTCTTAGAGTATCCAGTG |
| 11.2 | SaCC SEQ | SpCC-XL | SEQ ID NO: 13 |
| | ID NO: 42 | SEQ ID NO: | AAGGCAAGGCTGGCCAACCC |
| | | 41 | SEQ ID NO: 3 |
| | | | TAGTCTTAGAGTATCCAGTG |
| 12 | SaCC SEQ | SpCC | SEQ ID NO: 7 |
| | ID NO: 42 | SEQ ID NO: | AAGGCTGGCCAACCCATGGG |
| | | 39 | SEQ ID NO: 3 |
| | | | TAGTCTTAGAGTATCCAGTG |
| 12.1 | SaCC-XL | SpCC-XL | SEQ ID NO: 7 |
| | SEQ ID NO: | SEQ ID NO: | AAGGCTGGCCAACCCATGGG |
| | 43 | 41 | SEQ ID NO: 3 |
| | | | TAGTCTTAGAGTATCCAGTG |
| 12.2 | SaCC SEQ | SpCC-XL | SEQ ID NO: 7 |
| | ID NO: 42 | SEQ ID NO: | AAGGCTGGCCAACCCATGGG |
| | | 41 | SEQ ID NO: 3 |
| | | | TAGTCTTAGAGTATCCAGTG |

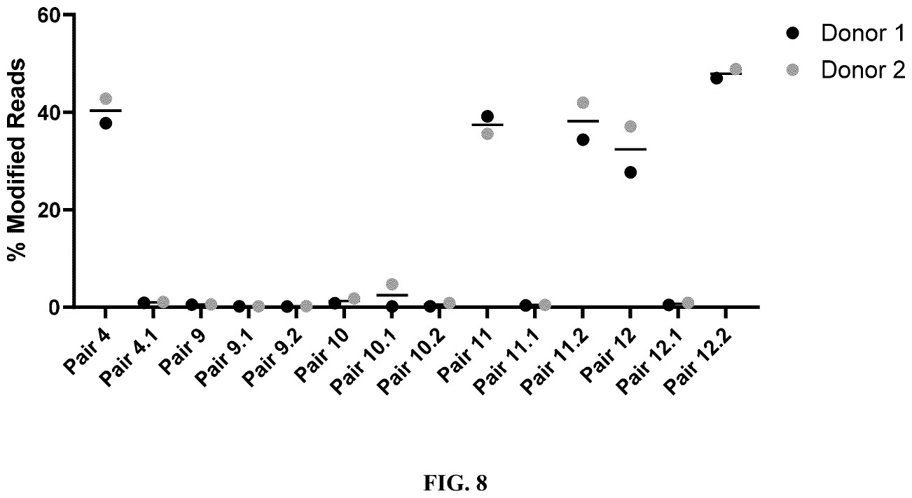

The results of this experiment is shown in FIG. 8. gRNA Pairs 11, 11.2, 12 and 12.2 demonstrated appreciable editing of the HBG loci resulting in indel percentages between about 25-50% comparable to or better than the benchmark Pair ID 4 in Table 8.

### Example 4: HBG Edited HSPCs Maintain Multi-lineage Differentiation Potential and Increase HBG mRNA and HbF Protein Levels

HSPCs were nucleofected with 200 μg/ml of Cas-CLOVER mRNA and with 400 μg/ml of each pair of gRNA Pair #2, #3 or #4 according to the methods described above. Four days later, 1×10⁵ electroporated cells were isolated and gDNA was extracted essentially according to the methods described above. The remaining HPSCs were divided into two separate groups: the first group was switched to erythroid differentiation as described in in the methods above; and the second group was subjected to the colony-forming unit (CFU) assay on MethoCult-based culture essentially as described in the methods above.

### A. Indels Via Next Generation Sequencing (NGS)

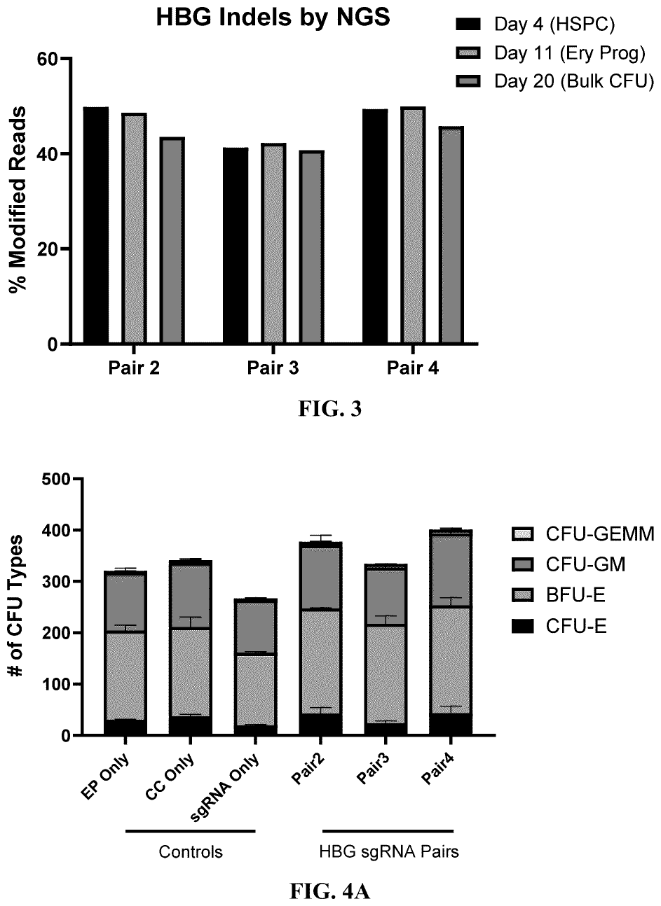

Induced indels from HBG editing of HSPCs, erythroid progenitors differentiated from edited HSPCs, and bulk colonies collected at the end of the CFU assay were measured via NGS and quantified by CRISPResso2 from cells at various time points after nucleofection according to the methods described above. At Day 4 HSPCs show robust editing at the HBG gene loci as evidenced by NGS sequencing with editing rates of approximately 40-50% of cells for all three gRNA pairs tested (FIG. 3). The percentage of HBG edited cells is maintained at Day 11 after erythroid differentiation, demonstrating that HBG edited HSPCs may be differentiated to erythroid progenitor cells which retain the edited HBG loci. In addition, bulk colonies collected at the end of the CFU assay also maintain similar editing percentages as HSPCs and erythroid progenitor cells demonstrating the HBG editing is maintained after HSPCs are differentiated down the erythropoiesis lineage.

### B. Colony-Forming Unit Assay

At Day 20 the absolute number of CFU cell types for HSPC control samples and for HBG edited HSPCs using gRNA Pairs #2, #3 or #4 are shown (FIG. 4A). The absolute number of cell types CFU-GEMM (CFU-Granulocyte/Erythrocyte/Macrophage/Megakaryocyte), CFU-GM (CFU-Granulocyte/Macrophage), BFU-E (Burst-forming Unit-Erythroid); and CFU-E (CFU-Erythroid) was unchanged in the CFU assay for HSPCs edited using the three HBG sgRNA pairs, as compared to three controls: EP only (nucleofection/electroporation only), CC only (Cas-CLOVER mRNA only) and sgRNA only. Moreover, the relative distribution of the CFU cell types also remained unchanged between controls and HBG edited cells using Pairs #2, #3 or #4 (FIG. 4B), demonstrating that editing using the compositions of the present disclosure do not affect HSPC multi-lineage colony forming capabilities.

C. HBG mRNA Expression in Edited HSPCs by RT-qPCR

HBG mRNA expression was measured by RT-qPCR according to the methods described above and normalized to HBB at Days 14, 18 and 21 during erythroid differentiation. Adult and umbilical cord blood samples were used as negative and positive controls, respectively. Fold changes were calculated by normalizing the observed expression levels to that of the EP only control for each of the respective time points.

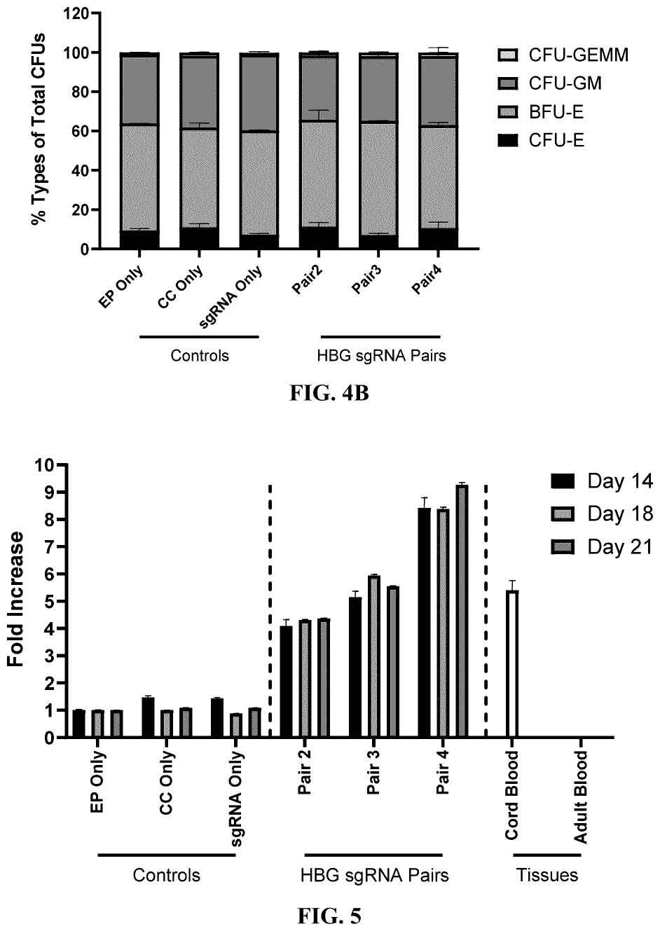

Increased HBG mRNA expression was observed in HSPC cells edited using HBG gRNA Pairs #2, #3 and #4 at all tested time points, with HBG mRNA expression increasing from Day 14 through Day 21, from about 4-fold to about 9-fold for cells edited using each of the gRNA Pairs (FIG. 5).

### D. HbF Protein Expression Levels

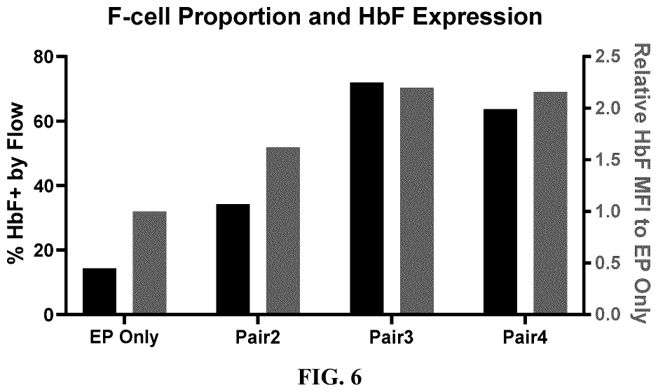

HbF protein was detected by intracellular flow cytometry of control and erythroid differentiated cells from HBG edited HSPCs according to the methods described above. The percentage of F-cells, as defined by HbF positive staining (HbF+), determined from intracellular flow cytometry is plotted on the left y-axis (FIG. 6). All three gRNA pairs demonstrated increased numbers of HbF positive cells compared to EP control with a range of about 2-fold to about 5-fold increase in the percentage of HbF positive cells.

The median fluorescence intensity (MFI) of HbF signal per F-cell was quantified relative to EP only control and plotted on the right y-axis (FIG. 6). The MFI for HBG edited F-cells was increased compared to EP control with Pair #3 demonstrating the highest MFI per F-cell amongst the three gRNA pairs tested.

What is claimed is:

1. A composition comprising
a) a first guide RNA (gRNA) and a first fusion protein or a first polynucleotide encoding the first fusion protein comprising: a mutant Cas9 (dCas9) polypeptide or an inactivated nuclease domain thereof and a Clo051 polypeptide or a nuclease domain thereof, configured to form a complex with the first gRNA, and
b) a second gRNA and a second fusion protein or a second polynucleotide encoding the second fusion protein comprising: a dCas9 polypeptide or an inactivated nuclease domain thereof and a Clo051 polypeptide or a nuclease domain thereof, configured to form a complex with the second gRNA,
wherein
i) the first gRNA comprises a first targeting sequence comprising a nucleotide sequence selected from SEQ ID NOs: 1, 5, 7, 13, 15 or 17; and
the second gRNA comprises a second targeting sequence comprising a nucleotide sequence of SEQ ID NO: 3, or
ii) the first gRNA comprises a first targeting sequence comprising a nucleotide sequence selected from SEQ ID NOs: 7, 9 or 13; and
the second gRNA comprises a second targeting sequence comprising a nucleotide sequence of SEQ ID NO: 11.

2. The composition of claim 1, wherein the first gRNA comprises a first scaffold sequence and the second gRNA comprises a second scaffold sequence, wherein the first scaffold sequence and the second scaffold sequence comprises a nucleotide sequence selected from SEQ ID NO: 20 or 21.

3. The composition of claim 2, wherein
a) the first gRNA comprises a nucleotide sequence selected from SEQ ID NOs: 2, 6, 8, 16 or 18; and the second gRNA comprises a nucleotide sequence of SEQ ID NO: 4, or
b) the first gRNA comprises a nucleotide sequence selected from SEQ ID NOs: 10, 14 or 19; and the second gRNA comprises a nucleotide sequence of SEQ ID NO: 12.

4. The composition of any one of claims 1-3, wherein the first gRNA, the second gRNA or both the first gRNA and the second gRNA comprises one or more chemical modifications of a ribonucleotide, a ribonucleotide base, or a phosphodiester bond.

5. The composition of claim 4, wherein the one or more chemical modification comprises at least one chemically modified phosphodiester bond.

6. The composition of claim 5, wherein the at least one chemically modified phosphodiester bond is a phosphorothioate bond.

7. The composition of claim 6, comprising at least two consecutive phosphorothioate bonds at the 5′-terminus of the first gRNA, the second gRNA or both the first gRNA and the second gRNA.

8. The composition of any one of claims 4-6, comprising a 2′ O-Me chemical modification at the 3′-terminus of the first gRNA, the second gRNA or both the first gRNA and the second gRNA.

9. The composition of any one of claims 1-8, wherein the dCas9 is derived from a S. pyogenes Cas9 polypeptide or a S. aureus Cas9 polypeptide.

10. The composition of any one of claims 1-8, wherein the C-terminus of the dCas9 or inactivated nuclease domain thereof, is joined to N-terminus of the Clo051 polypeptide or nuclease domain thereof via peptide linker sequence selected from GGGGS and SEQ ID NO: 23.

11. The composition of any one of claims 9-10, wherein first fusion protein comprises the amino acid sequence of SEQ ID NO: 39, 41, 42, or 43.

12. The composition of any one of claims 9-11, wherein the second fusion protein comprises the amino acid sequence of SEQ ID NO: 39, 41, 42, or 43.

13. The composition of any of claims 9-12, wherein the first polynucleotide, the second polynucleotide or both the first polynucleotide and the second polynucleotide are an mRNA.

14. The composition of claim 13, wherein the mRNA comprises a 5′-cap.

15. A composition comprising:
i) a first gRNA comprising a nucleotide sequence of SEQ ID NO: 2, a second gRNA comprising a nucleotide sequence of SEQ ID NO: 4, a first polynucleotide sequence encoding a first fusion protein of SEQ ID NO: 39 and a second polynucleotide sequence encoding a second fusion protein of SEQ ID NO: 39;
ii) a first gRNA comprising a nucleotide sequence of SEQ ID NO: 6, a second gRNA comprising a nucleotide sequence of SEQ ID NO: 4, a first polynucleotide sequence encoding a first fusion protein of SEQ ID NO: 39 and a second polynucleotide sequence encoding a second fusion protein of SEQ ID NO: 39;
iii) a first gRNA comprising a nucleotide sequence of SEQ ID NO: 8, a second gRNA comprising a nucleotide sequence of SEQ ID NO: 4, a first polynucleotide sequence encoding a first fusion protein of SEQ ID NO: 41 and a second polynucleotide sequence encoding a second fusion protein of SEQ ID NO: 41;
iv) a first gRNA comprising a nucleotide sequence of SEQ ID NO: 10, a second gRNA comprising a nucleotide sequence of SEQ ID NO: 12, a first polynucleotide sequence encoding a first fusion protein of SEQ ID NO: 39 and a second polynucleotide sequence encoding a second fusion protein of SEQ ID NO: 42;
v) a first gRNA comprising a nucleotide sequence of SEQ ID NO: 14, a second gRNA comprising a nucleotide sequence of SEQ ID NO: 12, a first polynucleotide sequence encoding a first fusion protein of SEQ ID NO: 42 and a second polynucleotide sequence encoding a second fusion protein of SEQ ID NO: 42;
vi) a first gRNA comprising a nucleotide sequence of SEQ ID NO: 19, a second gRNA comprising a nucleotide sequence of SEQ ID NO: 12, a first polynucleotide sequence encoding a first fusion protein of SEQ ID NO: 42 and a second polynucleotide sequence encoding a second fusion protein of SEQ ID NO: 42;
vii) a first gRNA comprising a nucleotide sequence of SEQ ID NO: 16, a second gRNA comprising a nucleotide sequence of SEQ ID NO: 4, a first polynucleotide sequence encoding a first fusion protein of SEQ ID NO: 41 and a second polynucleotide sequence encoding a second fusion protein of SEQ ID NO: 41; or
viii) a first gRNA comprising a nucleotide sequence of SEQ ID NO: 18, a second gRNA comprising a nucleotide sequence of SEQ ID NO: 4, a first polynucleotide sequence encoding a first fusion protein of SEQ ID NO: 41 and a second polynucleotide sequence encoding a second fusion protein of SEQ ID NO: 41.

16. The composition of any one of claims 1-15, wherein the composition is encapsulated in at least one lipid nanoparticle (LNP) comprising:
about 4.75% of a compound of Formula (I) by moles, 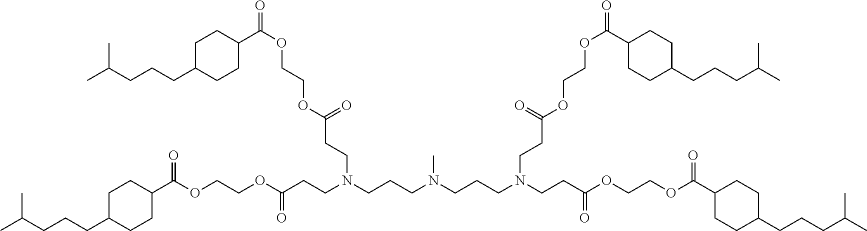 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]
about 51.75% of cholesterol by moles,
about 5% of DOPC by moles, and
about 2.5% of DMG-PEG2000 by moles;
wherein the first polynucleotide and the second polynucleotide is a RNA molecule, and wherein the ratio of lipid to RNA molecule in the at least one nanoparticle is about 120:1 (w/w).

17. The composition of any one of claims 1-15, wherein the composition is encapsulated in at least one LNP comprising:
about 54% of SS-OP by moles, about 35% of cholesterol by moles, about 5% of DOPC by moles, about 5% of DSPC by moles, and about 1% of DMG-PEG2000 by moles,
wherein the first polynucleotide and the second polynucleotide is a RNA molecule, and wherein the ratio of lipid to RNA molecule in the at least one nanoparticle is about 100:1 (w/w) and the total lipid of 25 nM.

18. The composition of any one of claims 1-17, for use in modifying a HBG1 gene, a HBG2 gene, a BCL11A gene or a combination thereof in a cell.

19. A method of modifying a population of cells comprising contacting the population of cells with the composition of any one of claims 1-18,
wherein the first gRNA forms a complex with the first targeting sequence and the first fusion protein, and the second gRNA forms a complex with the second targeting sequence and the second fusion protein,
thereby generating an indel between the first targeting sequence and the second targeting sequence and producing a modified population of cells.

20. The method of claim 19, wherein the indel causes inactivation of a BCL11A gene.

21. The method of any one of claims 19-20, wherein the modified population of cells have about 4-fold to about 9-fold increase in the expression of gamma globulin relative to an unmodified population of cells.

22. The method of any one of claims 19-20, wherein the modified population of cells have an increased level of fetal hemoglobin (HbF) expression relative to an unmodified population of cells.

23. The method of any one of claims 19-21, wherein the cells are hematopoietic stem and precursor cells (HSPCs).

24. The method of claim 23, wherein the HSPCs are capable of differentiating into erythroid progenitor cells.

25. A population of cells modified according to the method of any one of claims 19-24.

26. A method of treating a beta-hemoglobinopathy in a subject in need thereof, comprising administering to a subject the composition of any one of claims 1-18 or the population of cells of claim 25.

27. The method of claim 26, wherein the beta-hemoglobinopathy is beta-thalassemia or sickle cell disease.


SEQUENCE LISTING (43 sequences)

SEQ ID NO: 1 - RNA, 20 nt, synthetic construct
  source 1..20: mol_type = other RNA
gctattggtcaaggcaaggc

SEQ ID NO: 2 - RNA, 100 nt, synthetic construct
  source 1..100: mol_type = other RNA
gctattggtcaaggcaaggcgttttagagctagaaatagcaagttaaaataaggctagtccgttatcaacttgaaaaagtggcaccgagtcggtgctttt

SEQ ID NO: 3 - RNA, 20 nt, synthetic construct
  source 1..20: mol_type = other RNA
tagtcttagagtatccagtg

SEQ ID NO: 4 - RNA, 100 nt, synthetic construct
  source 1..100: mol_type = other RNA
tagtcttagagtatccagtggttttagagctagaaatagcaagttaaaataaggctagtccgttatcaacttgaaaaagtggcaccgagtcggtgctttt

SEQ ID NO: 5 - RNA, 20 nt, synthetic construct
  source 1..20: mol_type = other RNA
caaggctattggtcaaggca

SEQ ID NO: 6 - RNA, 100 nt, synthetic construct
  source 1..100: mol_type = other RNA
caaggctattggtcaaggcagttttagagctagaaatagcaagttaaaataaggctagtccgttatcaacttgaaaaagtggcaccgagtcggtgctttt

SEQ ID NO: 7 - RNA, 20 nt, synthetic construct
  source 1..20: mol_type = other RNA
aaggctggccaacccatggg

SEQ ID NO: 8 - RNA, 100 nt, synthetic construct
  source 1..100: mol_type = other RNA
aaggctggccaacccatggggttttagagctagaaatagcaagttaaaataaggctagtccgttatcaacttgaaaaagtggcaccgagtcggtgctttt

SEQ ID NO: 9 - RNA, 20 nt, synthetic construct
  source 1..20: mol_type = other RNA
ggcaaggctggccaacccat

SEQ ID NO: 10 - RNA, 100 nt, synthetic construct
  source 1..100: mol_type = other RNA
ggcaaggctggccaacccatgttttagagctagaaatagcaagttaaaataaggctagtccgttatcaacttgaaaaagtggcaccgagtcggtgctttt

SEQ ID NO: 11 - RNA, 20 nt, synthetic construct
  source 1..20: mol_type = other RNA
gcaaacttgaccaatagtct

SEQ ID NO: 12 - RNA, 100 nt, synthetic construct
  source 1..100: mol_type = other RNA
gcaaacttgaccaatagtctgttttagtactctggaaacagaatctactaaaacaaggcaaaatgccgtgtttatctcgtcaacttgttggcgagatttt

SEQ ID NO: 13 - RNA, 20 nt, synthetic construct
  source 1..20: mol_type = other RNA
aaggcaaggctggccaaccc

SEQ ID NO: 14 - RNA, 100 nt, synthetic construct
  source 1..100: mol_type = other RNA
aaggcaaggctggccaacccgttttagtactctggaaacagaatctactaaaacaaggcaaaatgccgtgtttatctcgtcaacttgttggcgagatttt

SEQ ID NO: 15 - RNA, 20 nt, synthetic construct
  source 1..20: mol_type = other RNA
cccatgggtggagtttagcc

SEQ ID NO: 16 - RNA, 100 nt, synthetic construct
  source 1..100: mol_type = other RNA
cccatgggtggagtttagccgttttagagctagaaatagcaagttaaaataaggctagtccgttatcaacttgaaaaagtggcaccgagtcggtgctttt

SEQ ID NO: 17 - RNA, 20 nt, synthetic construct
  source 1..20: mol_type = other RNA
ccatgggtggagtttagcca

SEQ ID NO: 18 - RNA, 100 nt, synthetic construct
  source 1..100: mol_type = other RNA
ccatgggtggagtttagccagttttagagctagaaatagcaagttaaaataaggctagtccgttatcaacttgaaaaagtggcaccgagtcggtgctttt

SEQ ID NO: 19 - RNA, 100 nt, synthetic construct
  source 1..100: mol_type = other RNA
aaggctggccaacccatggggttttagtactctggaaacagaatctactaaaacaaggcaaaatgccgtgtttatctcgtcaacttgttggcgagatttt

SEQ ID NO: 20 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 21 - RNA, 80 nt, synthetic construct
  source 1..80: mol_type = other RNA
gttttagtactctggaaacagaatctactaaaacaaggcaaaatgccgtgtttatctcgtcaacttgttggcgagatttt

SEQ ID NO: 22 - skipped

SEQ ID NO: 23 - RNA, 25 nt, synthetic construct
  source 1..25: mol_type = other RNA
ggggsggggsggggsggggsggggs

SEQ ID NO: 24 - DNA, 51 bp, synthetic construct
  source 1..51: mol_type = other DNA
acactctttccctacacgacgctcttccgatctgcagtatcctcttggggg

SEQ ID NO: 25 - DNA, 52 bp, synthetic construct
  source 1..52: mol_type = other DNA
gactggagttcagacgtgtgctcttccgatctacctcagacgttccagaagc

SEQ ID NO: 26 - DNA, 20 bp, synthetic construct
  source 1..20: mol_type = other DNA
tccacagtacctgccaaaga

SEQ ID NO: 27 - DNA, 20 bp, synthetic construct
  source 1..20: mol_type = other DNA
gcctaccttcccagggtttc

SEQ ID NO: 28 - DNA, 22 bp, synthetic construct
  source 1..22: mol_type = other DNA
ggcctaaaaccacagagagtat

SEQ ID NO: 29 - DNA, 20 bp, synthetic construct
  source 1..20: mol_type = other DNA
ccccacaggcttgtgatagt

SEQ ID NO: 30 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 31 - protein, 1053 aa, synthetic construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 32 - protein, 1362 aa, Streptococcus pyogenes
XDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDQFYKVREINNYHHAHDAYLNAVVGTALIKKYPKLESEFVYGDYKVYDVRKMIAKSEQEIKATAKYFFYSNIMNFFKTEITLANGEIRKRPLIETNGETGEIVWDKGRDFATVRKVLSMQVNIVKKTEVQTGGFSKESILPKRNSDKLIARKKDWDPKKYGGFDSPTVAYSVLVVAKVKGKSKKLKSVKELLGITIMERSSFEKNPIDFLEAKGYKEVKKDLIIKLPKYSLFELENGKRMLASAGELQKGNELALPSKYVNFLYLASHYEKLKGSPEDNEQKQLFVEQHKHYLDEIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 33 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 34 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 35 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 36 - DNA, 4779 bp, synthetic construct
  source 1..4779: mol_type = other DNA
atggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataa

SEQ ID NO: 37 - protein, 187 aa, synthetic construct
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 38 - DNA, 561 bp, synthetic construct
  source 1..561: mol_type = other DNA
atggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgac

SEQ ID NO: 39 - protein, 1591 aa, synthetic construct; identical to SEQ ID NO: 35

SEQ ID NO: 40 - DNA, 4779 bp, synthetic construct
  source 1..4779: mol_type = other DNA
atggcaccaaagaagaaaagaaaagtggagggcatcaagtcaaacatcagcctgctgaaagacgaactgcggggacagattagtcacatcagtcacgagtacctgtcactgattgatctggccttcgacagcaagcagaatagactgtttgagatgaaagtgctggaactgctggtcaacgagtatggcttcaagggcagacatctgggcgggtctaggaaacctgacggcatcgtgtacagtaccacactggaagacaacttcggaatcattgtcgataccaaggcttattccgagggctactctctgccaattagtcaggcagatgagatggaaaggtacgtgcgcgaaaactcaaatagggacgaggaagtcaaccccaataagtggtgggagaatttcagcgaggaagtgaagaaatactacttcgtctttatctcaggcagcttcaaagggaagtttgaggaacagctgcggagactgtccatgactaccggggtgaacggatctgctgtcaacgtggtcaatctgctgctgggcgcagaaaagatcaggtccggggagatgacaattgaggaactggaacgcgccatgttcaacaattctgagtttatcctgaagtatggaggcgggggaagcgataagaaatactccatcggactggccattggcaccaattccgtgggctgggctgtcatcacagacgagtacaaggtgccaagcaagaagttcaaggtcctggggaacaccgatcgccacagtatcaagaaaaatctgattggagccctgctgttcgactcaggcgagactgctgaagcaacccgactgaagcggactgctaggcgccgatatacccggagaaaaaatcggatctgctacctgcaggaaattttcagcaacgagatggccaaggtggacgatagtttctttcaccgcctggaggaatcattcctggtggaggaagataagaaacacgagcggcatcccatctttggcaacattgtggacgaagtcgcttatcacgagaagtaccctactatctatcatctgaggaagaaactggtggactccaccgataaggcagacctgcgcctgatctatctggccctggctcacatgatcaagttccgggggcattttctgatcgagggagatctgaaccctgacaattctgatgtggacaagctgttcatccagctggtccagacatacaatcagctgtttgaggaaaacccaattaatgcctcaggcgtggacgcaaaggccatcctgagcgccagactgtccaaatctaggcgcctggaaaacctgatcgctcagctgccaggagagaagaaaaacggcctgtttgggaatctgattgcactgtccctgggcctgacacccaacttcaagtctaattttgatctggccgaggacgctaagctgcagctgtccaaagacacttatgacgatgacctggataacctgctggctcagatcggcgatcagtacgcagacctgttcctggccgctaagaatctgagtgacgccatcctgctgtcagatattctgcgcgtgaacacagagattactaaggccccactgagtgcttcaatgatcaaaagatatgacgagcaccatcaggatctgaccctgctgaaggctctggtgaggcagcagctgcccgagaaatacaaggaaatcttctttgatcagagcaagaatggatacgccggctatattgacggcggggcttcccaggaggagttctacaagttcatcaagcccattctggaaaagatggacggcaccgaggaactgctggtgaagctgaatcgggaggacctgctgagaaaacagaggacatttgataacggaagcatccctcaccagattcatctgggcgaactgcacgccatcctgcgacggcaggaggacttctacccatttctgaaggataaccgcgagaaaatcgaaaagatcctgaccttcagaatcccctactatgtggggcctctggcacggggaaatagtagatttgcctggatgacaagaaagtcagaggaaactatcaccccctggaacttcgaggaagtggtcgataaaggcgctagcgcacagtccttcattgaaaggatgacaaattttgacaagaacctgccaaatgagaaggtgctgcccaaacacagcctgctgtacgaatatttcacagtgtataacgagctgactaaagtgaagtacgtcaccgaagggatgcgcaagcccgcattcctgtccggagagcagaagaaagccatcgtggacctgctgtttaagacaaatcggaaagtgactgtcaaacagctgaaggaagactatttcaagaaaattgagtgtttcgattcagtggaaatcagcggcgtcgaggacaggtttaacgcctccctggggacctaccacgatctgctgaagatcatcaaggataaggacttcctggacaacgaggaaaatgaggacatcctggaggacattgtgctgacactgactctgtttgaggatcgcgaaatgatcgaggaacgactgaagacttatgcccatctgttcgatgacaaagtgatgaagcagctgaaaagaaggcgctacaccggatggggacgcctgagccgaaaactgatcaatgggattagagacaagcagagcggaaaaactatcctggactttctgaagtccgatggcttcgccaacaggaacttcatgcagctgattcacgatgactctctgaccttcaaggaggacatccagaaagcacaggtgtctggccagggggacagtctgcacgagcatatcgcaaacctggccggcagccccgccatcaagaaagggattctgcagaccgtgaaggtggtggacgaactggtcaaggtcatgggacgacacaaacctgagaacatcgtgattgagatggcccgcgaaaatcagacaactcagaagggccagaaaaacagtcgagaacggatgaagagaatcgaggaaggcatcaaggagctggggtcacagatcctgaaggagcatcctgtggaaaacactcagctgcagaatgagaaactgtatctgtactatctgcagaatggacgggatatgtacgtggaccaggagctggatattaacagactgagtgattatgacgtggatgccatcgtccctcagagcttcctgaaggatgactccattgacaacaaggtgctgaccaggtccgacaagaaccgcggcaaatcagataatgtgccaagcgaggaagtggtcaagaaaatgaagaactactggaggcagctgctgaatgccaagctgatcacacagcggaaatttgataacctgactaaggcagaaagaggaggcctgtctgagctggacaaggccggcttcatcaagcggcagctggtggagacaagacagatcactaagcacgtcgctcagattctggatagcagaatgaacacaaagtacgatgaaaacgacaagctgatcagggaggtgaaagtcattactctgaaatccaagctggtgtctgactttagaaaggatttccagttttataaagtcagggagatcaacaactaccaccatgctcatgacgcatacctgaacgcagtggtcgggaccgccctgattaagaaataccccaagctggagtccgagttcgtgtacggagactataaagtgtacgatgtccggaagatgatcgccaaatctgagcaggaaattggcaaggccaccgctaagtatttcttttacagtaacatcatgaatttctttaagaccgaaatcacactggcaaatggggagatcagaaaaaggcctctgattgagaccaacggggagacaggagaaatcgtgtgggacaagggaagggattttgctaccgtgcgcaaagtcctgtccatgccccaagtgaatattgtcaagaaaactgaagtgcagaccgggggattctctaaggagagtattctgcctaagcgaaactctgataaactgatcgcccggaagaaagactgggaccccaagaagtatggcgggttcgactctccaacagtggcttacagtgtcctggtggtcgcaaaggtggaaaaggggaagtccaagaaactgaagtctgtcaaagagctgctgggaatcactattatggaacgcagctccttcgagaagaatcctatcgattttctggaagccaagggctataaagaggtgaagaaagacctgatcattaagctgccaaaatactcactgtttgagctggaaaacggacgaaagcgaatgctggcaagcgccggagaactgcagaagggcaatgagctggccctgccctccaaatacgtgaacttcctgtatctggctagccactacgagaaactgaaggggtcccctgaggataacgaacagaagcagctgtttgtggagcagcacaaacattatctggacgagatcattgaacagatttcagagttcagcaagagagtgatcctggctgacgcaaatctggataaagtcctgagcgcatacaacaagcaccgagacaaaccaatccgggagcaggccgaaaatatcattcatctgttcaccctgacaaacctgggcgcccctgcagccttcaagtattttgacaccacaatcgatcggaagagatacacttctaccaaagaggtgctggatgctaccctgatccaccagagtattaccggcctgtatgagacacgcatcgacctgtcacagctgggaggcgatgggagccccaagaaaaagcggaaggtgtctagttaatga

SEQ ID NO: 41 - protein, 1611 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSGGGGSGGGGSGGGGSGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 42 - protein, 1276 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGSPKKKRKVSS

SEQ ID NO: 43 - protein, 1296 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSGGGGSGGGGSGGGGSGGGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGSPKKKRKVSS